(19) United States
(12) Patent Application Publication
(10) Pub. No.: US 2012/0115907 A1
(43) Pub. Date: May 10, 2012

(54) NOVEL COMPOUNDS AS INHIBITORS OF RENIN

(75) Inventors: Pravain Thombare, Gujarat (IN); Jigar Desai, Gujarat (IN); Mukul R. Jain, Gujarat (IN)

(73) Assignee: CADILA HEALTHCARE LIMITED, Ahmedabad, Gujarat (IN)

(21) Appl. No.: 13/265,537

(22) PCT Filed: Apr. 22, 2010
(86) PCT No.: PCT/IN2010/000253

§ 371 Date: Dec. 28, 2011

(30) Foreign Application Priority Data
Apr. 24, 2009 (IN) 1091/MUM/2009

(51) Int. Cl. A61K 31/4468; C07D 401/06; A61K 31/4545; C07D 405/06; A61K 31/453; A61P 9/12; A61P 9/04; A61P 13/12; A61P 9/00; A61P 9/10; A61P 7/00; A61P 25/00; A61P 27/06; A61P 27/02; A61P 15/10; A61P 5/40; A61P 11/00; A61P 17/00; A61P 25/22; A61P 25/28; C07D 211/58

(52) U.S. Cl. 514/316; 546/224; 514/329; 546/189; 546/197; 514/321

(57) ABSTRACT

The present invention relates to novel renin inhibitors of general formula (1), novel intermediates involved in their synthesis, their pharmaceutically acceptable salts and pharmaceutical compositions containing them. The present invention also relates to a process of preparing compounds of general formula (1), their tautomeric forms, their pharmaceutically acceptable salts, pharmaceutical compositions containing them, and novel intermediates involved in their synthesis.

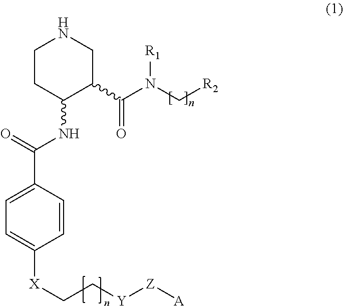

### FIELD OF INVENTION

The present invention relates to novel renin inhibitors of general formula (1), novel intermediates involved in their synthesis, their pharmaceutically acceptable salts and pharmaceutical compositions containing them. The present invention also relates to a process of preparing compounds of general formula (1), their tautomeric forms, their pharmaceutically acceptable salts, pharmaceutical compositions containing them, and novel intermediates involved in their synthesis.

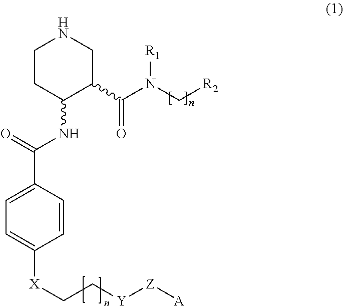

### BACKGROUND OF THE INVENTION

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT1 and AT2. Whereas AT1 seems to transmit most of the known functions of Ang II, the role of AT2 is still unknown.

Modulation of the RAS represents a major advance in the treatment of cardiovascular diseases. ACE inhibitors and AT1 blockers have been accepted to treat hypertension (Waeber B. et al, “The renin-angiotensin system: role in experimental and human hypertension”, in Birkenhager W. H., Reid J. L. (eds): Hypertension, Amsterdam, Elsevier Science Publishing Co, 1986, 489-519; Weber M A., Am. J. Hypertens., 1992, 5, 2478). In addition, ACE inhibitors are used for renal protection (Rosenberg M. E. et al, Kidney International, 1994, 45, 403; Breyer J. A. et al. Kidney International, 1994, 45, S156), in the prevention of congestive heart failure (Vaughan D. E. et al, Cardiovasc. Res., 1994, 28, 159; Fouad-Tarazi F. et al., Am. J. Med, 1988, 84 (Suppl. 3A), 83) and myocardial infarction (Pfeffer M. A. et al, N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1 155). In patients inhibition of ACE thus leads to bradykinin accumulation causing cough (5-20%) and potentially life-threatening angioneurotic edema (0.1-0.2%) (Israili Z. H. et al, Annals of Internal Medicine, 1992, 117, 234). Chymase is not inhibited by ACE inhibitors. Therefore, the formation of Ang II is still possible in patients treated with ACE inhibitors. Blockade of the AT1 receptor (e.g. by losartan) on the other hand overexposes other AT-receptor subtypes (e.g. AT₂) to Ang II, whose concentration is significantly increased by the blockade of AT1 receptors. In summary, renin inhibitors are expected to demonstrate a different pharmaceutical profile than ACE inhibitors and AT1 blockers with regard to efficacy in blocking the RAS and in safety aspects. Only limited clinical experience (Azizi M. et al., J. Hypertens., 1994, 12, 419; Neutel J. M. et al, Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. Only few compounds containing three to four chiral centers has entered clinical trials (Rahuel J. et al. Chem. Biol., 2000, 7, 493; Mealy N. E., Drugs of the Future, 2001, 26, 1139). Thus, renin inhibitors with good oral bioavailability and long duration of action are required. The first non-peptide renin inhibitors were described which show high in vitro activity (Oefner C. et al, Chem. Biol, 1999, 6, 127; Patent Application WO97/0931 1; Marki H. P. et al., 11 Farmaco, 2001, 56, 21).

The present invention relates to the identification of renin inhibitors of a non-peptidic nature and of low molecular weight. Described are orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis.

### SUMMARY OF THE INVENTION

The present invention describes a group of novel compounds as Renin inhibitors useful for the treatment cardiovascular events, renal insufficiency and other related diseases. The novel compounds are defined by the general formula (1) below:

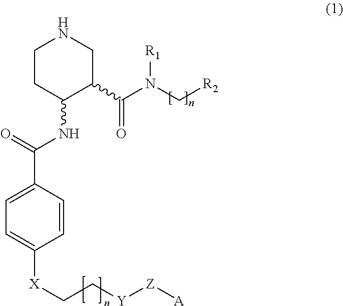

The compounds of the present invention are useful in the treatment of the human or animal body, by regulating Renin levels. The compounds of this invention are therefore suitable for the treatment of cardiovascular events, renal insufficiency other related diseases. Disclosed herein are also processes for preparing the compounds of formula (I) and also suitable pharmaceutical compositions containing the compounds of the present invention.

### EMBODIMENTS OF THE INVENTION

The main objective of the present invention thus is to provide novel compounds of general formula (1), novel intermediates involved in their synthesis, their pharmaceutically acceptable salts, and pharmaceutical compositions containing them or their mixtures as therapeutic agents.

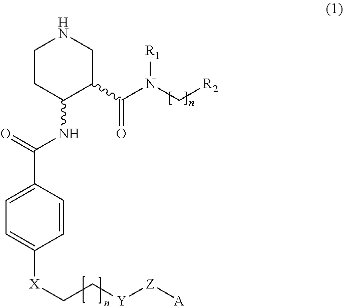

In an embodiment is provided processes for the preparation of novel compounds of general formula (1), novel intermediates involved in their synthesis, their pharmaceutically acceptable salts, and pharmaceutical compositions containing them.

In another embodiment is provided pharmaceutical compositions containing compounds of general formula (1), their pharmaceutically acceptable salts, comprising pharmaceutically acceptable carriers, solvents, diluents, excipients and other media normally employed in their manufacture.

In a further embodiment is provided the use of the novel compounds of the present invention as blood pressure regulating agents, by administering a to therapeutically effective & non-toxic amount of the compounds of formula (1) or their pharmaceutically acceptable compositions to the mammals.

### DETAILED DESCRIPTION OF THE INVENTION

The novel compounds of the present invention are defined by the general formula (1) below:

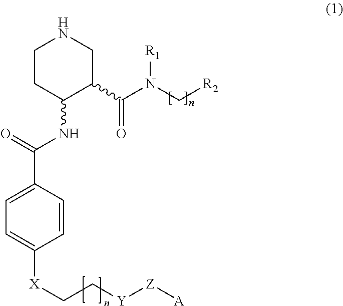

wherein
Z represents either a bond or —CH₂—;
X and Y are each independently selected from the group comprising of —CH₂—, O, and
S(O)ₚ; p in each instance when it occurs is independently selected from the integers 0,1,2;
n is an integer selected from 0,1,2;
‘A’ is an optionally substituted aryl or a heteroaryl ring wherein the heteroaryl group contains from 1 to 3 heteroatoms selected from O, S, and N; in an embodiment the group representing ‘A’ may be further substituted with groups independently selected from OH, CN, halogen, N₃, NO₂, COOH, OCF₂H, CF₃, C₍₁₋₆₎ alkyl, C₍₂₋₆₎ alkenyl, C₍₁₋₆₎ alkoxy, C(O)C₁-C₆ alkyl, S(O)ₚ C₍₁₋₆₎ alkyl or (CH₂)₁₋₂O-alkyl groups;
R₁ is optionally substituted C₁₋C₆ alkyl, or C₃-C₇ cycloalkyl groups;
R₂ represents an aryl, or a heteroaryl group, or a hetrocycle group, wherein the both heteroaryl group and heterocycle group may contain from 1 to 3 heteroatoms selected from O, S, and N & wherein each of the said groups can be optionally substituted with substituents independently selected from the group consisting of alkyl; halogen; alkoxy; —OCF₃, CF₃, hydroxyl-alkyl; alkyl-O—(CH₂)₀₋₄—CH₂—; alkyl-O—(CH₂)₂₋₄—O—; (R₃)₂N—(CH₂)₀₋₄—CH₂—; wherein R₃ is independently selected from the group comprising of hydrogen, alkyl groups wherein the alkyl group may be optionally substituted with one, two or three halogen atoms, optionally substituted cycloalkyl, or the groups selected from —C(═O)OR₄ or —C(O)R₄, wherein R₄ represents optionally substituted groups selected from (C₁-C₄) alkyl, (C₁-C₄)haloalkyl or a cycloalkyl or the group representing R₅NH—C(═O)—(O)₀₋₁—(CH₂)₀₋₄—CH₂₋ wherein R₅ is an optionally substituted alkyl or cycloalkyl group.

In a further embodiment, R₁ and R₂ together with the nitrogen atom attached to R₁ may together form a saturated, unsaturated or partly saturated single or fused heterocyclic ring which may optionally contain one or more additional hetero atoms selected from nitrogen, oxygen or sulphur or may comprise an —SO— or an —SO₂-group. When the heterocyclic ring as defined above further comprises one or more nitrogen atom, such nitrogen atom may optionally be substituted with optionally substituted groups selected from C₁-C₈ alkyl, C₁-C₈ alkanoyl, aryl or heterocyclic group;

In a preferred embodiment, when R₁ and R₂ in combination represent a fused ring, then the ring system preferably forms 9-16 membered heterocyclic groups or hetroaryl group. The said heterocyclic or heteroarylgroup may optionally be substituted with groups selected from halogen, hydroxyl, oxide, oxo, cyano, optionally substituted groups selected from haloalkyl, haloalkoxy, C₁-C₈-alkyl, C₁-C₈-alkoxy, C₁-C₈-alkoxy-C₁-C₈-alkyl, C₁-C₈-alkoxy-C₁-C₈-alkoxy, C₁-C₈-alkoxycarbonylamino, C₁-C₈-alkylcarbonylamino, C₁-C₈-alkylamino, N,N-di-C₁-C₈-alkylamino, aryl-C₀-C₄-alkyl, aryloxy-C₀-C₄-alkyl, aryl-C₀-C₄-alkyl-C₁-C₈-alkoxy, aryloxy-C₀-C₄-alkyl-C₁-C₈-alkoxy, heterocyclyl-C₀-C₄-alkyl, heterocyclyloxy-C₀-C₄-alkyl, heterocyclyl-C₀-C₄-alkyl-C₁-C₈-alkoxy or heterocyclyloxy-C₀-C₄-alkyl-C₁-C₈-alkoxy groups.

In one embodiment the “Heterocyclyl” group refers to a stable 3- to 15-membered non-aromatic ring radical which consists of carbon atoms and from one to five heteroatoms selected from a group consisting of nitrogen, oxygen and sulfur. In one embodiment, the heterocyclic ring system radical may be a monocyclic, bicyclic or tricyclic ring or tetracyclic ring system, which may include fused or bridged ring systems; and the nitrogen or sulfur atoms in the heterocyclic ring system radical may be optionally oxidized; the nitrogen atom may be optionally quaternized; and the heterocyclyl radical may be partially or fully saturated. The heterocyclic ring system may be attached to the main structure at any heteroatom or carbon atom which results in the creation of a stable compound. If may be specifically noted, nitrogen in the heterocycle may optionally be quaternized. It is preferred that when the total number of to S and O atoms in the heterocycle exceeds 1, then these heteroatoms are not adjacent to one another. It is preferred that the total number of S and O atoms in the heterocycle is not more than 1 Exemplary heterocylic radicals include, azetidinyl, benzo[1,3]dioxol-5-yl, benzodioxolyl, 1,3-dioxolan-2-yl, dioxolanyl, morpholinyl, tetrahydrofuran, oxazolidin-2-onyl, oxazolidinonyl, piperidinyl, piperazinyl, pyranyl, tetrahydropyranyl, pyrrolidinonyl, oxathiolanyl, and pyrrolidinyl.

In another embodiment the “Heteroaryl” group refers to a heterocyclyl group as defined above which is aromatic. The heteroaryl group may be attached to the main structure at any heteroatom or carbon atom which results in the creation of a stable compound. Examples of such heteroaryl groups include, but are not limited to: acridinyl, benzimidazolyl, benzindolyl, benzisoxazinyl, benzo[4,6]imidazo[1,2-α]pyridinyl, benzofuranyl, benzonaphthofuranyl, benzothiadiazolyl, benzothiazolyl, benzothiophenyl, benzotriazolyl, benzothiopyranyl, benzoxazinyl, benzoxazolyl, benzothiazolyl, β-carbolinyl, carbazolyl, cinnolinyl, dibenzofuranyl, furanyl, imidazolyl, imidazopyridinyl, imidazothiazolyl, indazolyl, indol izinyl, indolyl, isobenzothienyl, isoindolinyl, isoquinolinyl, isothiazolidinyl, isothiazolyl, naphthyridinyl, octahydroindolyl, octahydroisoindolyl, oxazolidinonyl, oxazolidinyl, oxazolopyridinyl, oxazolyl, isoxazolyl, oxiranyl, perimidinyl, phenanthridinyl, phenathrolinyl, phenarsazinyl, phenazinyl, phenothiazinyl, phenoxazinyl, phthalazinyl, pteridinyl, purinyl, pyrazinyl, pyrazolyl, pyridazinyl, pyridinyl, pyridopyridinyl, pyrimidinyl, pyrrolyl, quinazolinyl, quinolinyl, quinoxalinyl, tetrazolyl, thiadiazolyl, thiazolyl, thienyl, triazinyl and triazolyl.

In certain embodiments, the heterocyclic or heteroaryl radicals include, but are not limited to: acridinyl, azepinyl, benzimidazolyl, benzindolyl, benzoisoxazolyl, benzisoxazinyl, benzo[4,6]imidazo[1,2-α]pyridinyl, benzodioxanyl, benzodioxolyl, benzofuranonyl, benzopyranyl, benzonaphthofuranyl, benzopyranonyl, benzopyranyl, benzotetrahydrofuranyl, benzotetrahydrothienyl, benzothiadiazolyl, benzothiazolyl, benzothiophenyl, benzotriazolyl, benzothiopyranyl, benzoxazinyl, benzoxazolyl, benzothiazolyl, β-carbolinyl, carbazolyl, chromanyl, chromonyl, cinnolinyl, coumarinyl, decahydroisoquinolinyl, dibenzofuranyl, dihydrobenzisothiazinyl, dihydrobenzisoxazinyl, dihydrofuryl, dihydropyranyl, dioxolanyl, dihydropyrazinyl, dihydropyridinyl, dihydropyrazolyl, dihydropyrimidinyl, dihydropyrrolyl, dioxolanyl, 1, 4-dithianyl, furanonyl, furanyl, imidazolidinyl, imidazolinyl, imidazolyl, imidazopyridinyl, imidazothiazolyl, indazolyl, indolinyl, indolizinyl, indolyl, to isobenzotetrahydrofuranyl, isobenzotetrahydrothienyl, isobenzothienyl, isochromanyl, isocoumarinyl, isoindolinyl, isoindolyl, isoquinolinyl, isothiazolidinyl, isothiazolyl, isoxazolidinyl, isoxazolyl, morpholinyl, naphthyridinyl, octahydroindolyl, octahydroisoindolyl, oxadiazolyl, oxazolidinonyl, oxazolidinyl, oxazolopyridinyl, oxazolyl, oxiranyl, perimidinyl, phenanthridinyl, phenathrolinyl, phenarsazinyl, phenazinyl, phenothiazinyl, phenoxazinyl, phthalazinyl, piperazinyl, piperidinyl, 4-piperidonyl, pteridinyl, purinyl, pyrazinyl, pyrazolidinyl, pyrazolyl, pyridazinyl, pyridinyl, pyridopyridinyl, pyrimidinyl, pyrrolidinyl, pyrrolinyl, pyrrolyl, quinazolinyl, quinolinyl, quinoxalinyl, quinuclidinyl, tetrahydrofuryl, tetrahydrofuranyl, tetrahydroisoquinolinyl, tetrahydropyranyl, tetrahydrothienyl, tetrazolyl, thiadiazolopyrimidinyl, thiadiazolyl, thiamopholinyl, thiazolidinyl, thiazolyl, thienyl, triazinyl, triazolyl and 1,3,5-trithianyl.

The term “substituted,” as used herein, means that any one or more hydrogens on the designated atom is replaced with a selection from the indicated group, provided that the designated atom's normal valency is not exceeded, and that the substitution results in a stable compound.

In preferred embodiment, the suitable substituent wherever applicable includes, but are not limited to the following radicals, alone or in combination with other radicals, hydroxyl, oxo, halo, thio, nitro, amino, alkyl, alkoxy, haloalkyl or haloalkoxy groups;

In a further embodiment the groups, radicals described above may be selected from:
the “alkyl” group used either alone or in combination with other radicals, denotes a linear or branched radical containing one to six carbons, selected from methyl, ethyl, n-propyl, iso-propyl, n-butyl, sec-butyl, t-butyl, amyl, t-amyl, n-pentyl, n-hexyl, and the like;
The term “alkenyl” used herein, either alone or in combination with other radicals, denotes a linear or branched radical containing two to twelve carbons; such as vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl and the like. The term “alkenyl” includes dienes and trienes of straight and branched chains;
The term “alkynyl” used herein, either alone or in combination with other radicals, denotes a linear or branched radical containing two to twelve carbons, such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 3-hexynyl, 4-hexynyl, 5-hexynyl, and the like. The term “alkynyl” includes di- and tri-ynes;
The term “alkoxy” used herein, either alone or in combination with other radicals, denotes a radical alkyl, as defined above, attached directly to an oxygen atom, such as methoxy, ethoxy, n-propoxy, iso-propoxy, n-butoxy, t-butoxy, iso-butoxy, pentyloxy, hexyloxy, and the like;
The term “halo” or “halogen” used herein, either alone or in combination with other radicals, such as “haloalkyl”, “perhaloalkyl” etc refers to a fluoro, chloro, bromo or iodo group. The term “haloalkyl” denotes an alkyl radical, as defined above, substituted with one or more halogens; such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups. The term “haloalkoxy” denotes a haloalkyl, as defined above, directly attached to an oxygen atom, such as fluoromethoxy, chloromethoxy, fluoroethoxy chloroethoxy groups, and the like.
The term “aryl” refers to aromatic mono- and poly-carbocyclic ring systems, wherein the individual carbocyclic rings in the polyring systems are fused or attached to each other via a single bond. Suitable aryl groups include phenyl, naphthyl, and biphenylenyl.

The term “C₀” as employed in expressions such as “C₀-C₄ alkyl” means a direct covalent bond. Similarly, when an integer defining the presence of certain number of atoms in a group is equal to zero, it means that the atom adjacent thereto is connected directly by a bond.

The compound of the present invention may have chiral centers, e.g. two chiral centers [providing up to four stereoisomers (R,R), (S,S), (R,S), (S,R)] or three chiral centers [providing up to eight stereoisomers]. This invention includes all the optical isomers and mixture thereof. Unless specifically mentioned otherwise, reference to one isomer applies to any of the possible isomers. Whenever the isomeric composition is unspecified, all the possible isomers are included.

The present invention also relates to pro-drugs of a compound of formula (1) that convert in vivo to the compound of formula (1) as such. Any reference to a compound of formula (1) is therefore to be understood as referring also to the corresponding pro-drugs of the compound of formula (1), as appropriate and expedient.

### LIST OF ABBREVIATION

Boc: t-butyloxycarbonyl
DMF: Dimethyl formamide
DMSO: Dimethyl sulfoxide

### THF: Tetrahydrofuran

### DCM: Dichloromethane

EDAC.HCl: N-(3-Dimethyl aminopropyl)-N′-ethyl carbodiimide hydrochloride,
EDC: 1,2-dichloro ethane
HOBT: 1-Hydroxy benzotriazole
TFA: Trifluoro acetic acid

### DCC: Dicyclohexylcarbodiimide

DIEA: Disopropyl ethyl amine
EtOAc: Ethyl acetate

### h: Hour(s)

min: minute(s)
t_{Ret}: Retention time
HCl: Hydrochloric acid
RT: room temperature [25-30° C.]

Suitable groups and substituents on the groups may be selected from those described anywhere in the specification.

Particularly useful compounds may be selected from
N-Cyclopropyl-N-(2,3-dichlorobenzyl)-4-(4-(2-(2,6-dichlorophenoxy)ethoxy)benzamido) piperidine-3-carboxamide.
4-(2-(2,6-Dichlorophenoxy)ethoxy)-N-(3-(piperidine-1-carbonyl)piperidin-4-yl)benzamide.
N-Cyclopropyl-4-(4-(2-(2,6-dichlorophenoxy)ethoxy)benzamido)-N-(3-(3-methoxypropoxy)benzyl)piperidine-3-carboxamide.
N-Cyclopropyl-4-(4-(2-(2,6-dichlorophenoxy)ethoxy)benzamido)-N-(3,5-dimethylbenzyl)piperidine-3-carboxamide.
N-Cyclopropyl-4-(4-(2-(2,6-dichloro-4-methylphenoxy)ethoxy)benzamido)-N-(2,4-dichlorobenzyl)piperidine-3-carboxamide.
N-Cyclopropyl-N-(2,4-dichlorobenzyl)-4-(4-(2-(2,6-dichlorophenoxy)ethoxy) to benzamido) piperidine-3-carboxamide.
N-Cyclopropyl-4-(4-(2-(2,6-dichlorophenoxy)ethoxy)benzamido)-N-(3,4-dimethoxybenzyl)piperidine-3-carboxamide [Non polar Isomer].
N-Cyclopropyl-4-(4-(2-(2,6-dichloro-4-methylphenoxy)ethoxy)benzamido)-N-(3,4-dimethoxybenzyl)piperidine-3-carboxamide [Polar isomer]
N-Cyclopropyl-4-(4-(2-(2,6-dichloro-4-methylphenoxy)ethoxy)benzamido)-N-(3,4-dimethoxybenzyl)piperidine-3-carboxamide [Non polar isomer].
N-Cyclopropyl-4-(4-(2-(2,6-dichloro-4-methylphenoxy)ethoxy)benzamido)-N-(3,5-dimethylbenzyl)piperidine-3-carboxamide.
N-Cyclopropyl-4-(4-(2-(2,6-dichlorophenoxy)ethoxy)benzamido)-N-(naphthalen-2-ylmethyl)piperidine-3-carboxamide.
N-Cyclopropyl-4-(4-(2-(2,6-dichloro-4-methylphenoxy)ethoxy)benzamido)-N-(naphthalen-2-ylmethyl)piperidine-3-carboxamide.
4-(2-(2,6-Dichloro-4-methylphenoxy)ethoxy)-N-(3-(3-(methoxymethyl)piperidine-1-carbonyl)piperidin-4-yl)benzamide.
(4S)—N-Cyclopropyl-4-(4-(2-(2,6-dichloro-4-methylphenoxy)ethoxy)benzamido)-N-(2,3-dimethoxybenzyl)piperidine-3-carboxamide hydrochloride (4S)—N-Cyclopropyl-4-(4-(2-(2,6-dichloro-4-methylphenoxy)ethoxy)benzamido)-N-(2,3-dimethoxybenzyl)piperidine-3-carboxamide hydrochloride.
(4S)—N-Cyclopropyl-N-(2,3-dimethylbenzyl)-4-(4-(2-((2-methoxybenzyl)oxy)ethoxy)benzamido)piperidine-3-carboxamide.
(4S)—N-Cyclopropyl-N-(2,3-dimethylbenzyl)-4-(4-(2-((2-methoxybenzyl)oxy)ethoxy)benzamido)piperidine-3-carboxamide.
(4S)—N-Cyclopropyl-4-(4-(2-(2,6-dichloro-4-methylphenoxy)ethoxy)benzamido)-N-(3,4-dimethylbenzyl)piperidine-3-carboxamide hydrochloride.
(4S)—N-Cyclopropyl-4-(4-(2-(2,6-dichloro-4-methylphenoxy)ethoxy)benzamido)-N-(2,3-dimethylbenzyl)piperidine-3-carboxamide hydrochloride.
(4S)—N-Cyclopropyl-4-(4-(2-(2,6-dichloro-4-methylphenoxy)ethoxy)benzamido)-N-(2,3-dimethylbenzyl)piperidine-3-carboxamide hydrochloride.
(4S)—N-Cyclopropyl-4-(4-(2-(2,6-dichloro-4-methylphenoxy)ethoxy)benzamido)-N-(3,4-dimethylbenzyl)piperidine-3-carboxamide hydrochloride.
(4R)—N-Cyclopropyl-4-(4-(2-(2,6-dichloro-4-methylphenoxy)ethoxy)benzamido)-N-(3,4-dimethylbenzyl)piperidine-3-carboxamide hydrochloride.
(4R)—N-Cyclopropyl-4-(4-(2-(2,6-dichloro-4-methylphenoxy)ethoxy)benzamido)-N-to (3,4-dimethylbenzyl)piperidine-3-carboxamide hydrochloride.
(4R)—N-Cyclopropyl-4-(4-(2-(2,6-dichloro-4-methylphenoxy)ethoxy)benzaido)-N-(2,3-dimethylbenzyl)piperidine-3-carboxamide hydrochloride.
(4R)—N-Cyclopropyl-4-(4-(2-(2,6-dichloro-4-methylphenoxy)ethoxy)benzaido)-N-(2,3-dimethylbenzyl)piperidine-3-carboxamide hydrochloride.
(4S)—N-Cyclopropyl-4-(4-(2-(2,6-dichloro-4-methylphenoxy)ethoxy)benzamido)-N-(2,3-dimethylbenzyl)piperidine-3-carboxamide.
(4S)—N-Cyclopropyl-N-(2,3-dimethylbenzyl)-4-(4-(3-((2-methoxybenzypoxy) propoxy)benzamido)piperidine-3-carboxamide hydrochloride [Non polar isomer].
(4S)—N-cyclopropyl-N-(2,3-dimethylbenzyl)-4-(4-(3-((2-methoxybenzyl)oxy)propoxy)benzamido)piperidine-3-carboxamide hydrochloride.
(4S)—N-Cyclopropyl-4-(4-(2-(2,6-dichloro-4-methyl-phenoxy)ethoxy)benzamido)-N-(2-fluorobenzyl)piperidine-3-carboxamide hydrochloride.
(4S)—N-(2-Chlorobenzyl)-N-cyclopropyl-4-(4-(2-(2,6-dichloro-4-methylphenoxy)ethoxy)benzamido)piperidine-3-carboxamide hydrochloride.
(4 S)—N-Cyclopropyl-4-(4-(2-(2,6-dichloro-4-methylphenoxy)ethoxy)benzamido)-N-(2-methoxybenzyl)piperidine-3-carboxamide hydrochloride.
(4S)—N-Cyclopropyl-4-(4-(2-(2,6-dichloro-4-methylphenoxy)ethoxy)benzamido)-N-(2-methoxybenzyl)piperidine-3-carboxamide hydrochloride [Non polar isomer].
(4S)—N-Cyclopropyl-N-(2-methoxybenzyl)-4-(4-(3-((2-methoxybenzyl)oxy)propoxy)benzamido)piperidine-3-carboxamide hydrochloride.
(4S)—N-Cyclopropyl-N-(2-methoxybenzyl)-4-(4-(342-methoxybenzypoxy)propoxy)benzamido)piperidine-3-carboxamide hydrochloride [Non polar isomer].
(4S)—N-Cyclopropyl-4-(4-(2-(2,6-dichloro-4-methylphenoxy)ethoxy)benzamido)-N-(2,3-dichlorobenzyl)piperidine-3-carboxamide hydrochloride.
(4S)—N-Cyclopropyl-4-(4-(2-(2,6-dichloro-4-methylphenoxy)ethoxy)benzamido)-N-(3-methoxy-2-methylbenzyl)piperidine-3-carboxamide hydrochloride [Non polar isomer].
(4S)—N-cyclopropyl-4-(4-(2-(2,6-dichloro-4-methylphenoxy)ethoxy)benzamido)-N-(3-methoxy-2-methylbenzyl)piperidine-3-carboxamide hydrochloride.
(4S)—N-Cyclopropyl-4-(4-(2-(2,6-difluorophenoxy)ethoxy)benzamido)-N-(2,3-dimethylbenzyl)piperidine-3-carboxamide hydrochloride.
(4S)—N-Cyclopropyl-4-(4-(2-(2,6-difluorophenoxy)ethoxy)benzamido)-N-(2,3-dimethylbenzyl)piperidine-3-carboxamide hydrochloride [Non polar isomer].
(4S)—N-Cyclopropyl-4-(4-(2-(2,6-dichloro-4-methylphenoxy)ethoxy)benzamido)-N-(2,3-dimethylbenzyl)piperidine-3-carboxamide methanesulfonate.
(4S)—N-Cyclopropyl-4-(4-(2-(2,6-dichloro-4-methylphenoxy)ethoxy)benzamido)-N-(2,3-dimethylbenzyl)piperidine-3-carboxamide 2,2,2-trifluoroacetate.
(4S)—N-Cyclopropyl-4-(4-(2-(2,6-dichloro-4-methylphenoxy)ethoxy)benzamido)-N-(2,3-dimethylbenzyl)piperidine-3-carboxamide bisulfate.
(4S)—N-cyclopropyl-4-(4-(2-(2,6-dichloro-4-methylphenoxy)ethoxy)benzamido)-N-((2,3-dihydrobenzo[b][1,4]dioxin-6-yl)methyl)piperidine-3-carboxamide hydrochloride [Non polar isomer].
(4S)—N-cyclopropyl-4-(4-(2-(2,6-dichloro-4-methylphenoxy)ethoxy)benzamido)-N-((2,3-dihydrobenzo[b][1,4]dioxin-6-yl)methyl)piperidine-3-carboxamide hydrochloride
(4S)—N-(benzo[d][1,3] dioxol-4-ylmethyl)-N-cyclopropyl-4-(4-(2-(2,6-dichloro-4-methylphenoxy)ethoxy)benzamido)piperidine-3-carboxamide hydrochloride [Non polar isomer].
(4S)—N-(benzo[d][1,3] dioxol-4-ylmethyl)-N-cyclopropyl-4-(4-(2-(2,6-dichloro-4-methylphenoxy)ethoxy)benzamido)piperidine-3-carboxamide hydrochloride.
(4S)—N-cyclopropyl-4-(4-(2-(2,6-dichloro-4-methylphenoxy)ethoxy)benzamido)-N-((2,3-dihydrobenzo[b][1,4] dioxin-5-yl)methyl)piperidine-3-carboxamide hydrochloride [Non polar isomer].
(4S)—N-cyclopropyl-4-(4-(2-(2,6-dichloro-4-methylphenoxy)ethoxy)benzamido)-N-((2,3-dihydrobenzo[b][1,4]dioxin-5-yl)methyl)piperidine-3-carboxamide hydrochloride [Polar isomer.
(4S)—N-cyclopropyl-4-(4-(2-(2,6-dichloro-4-methylphenoxy)ethoxy)benzamido)-N-((1-methoxynaphthalen-2-yl)methyl)piperidine-3-carboxamide hydrochloride [Non polar isomer].
(4S)—N-cyclopropyl-4-(4-(2-(2,6-dichloro-4-methylphenoxy)ethoxy)benzamido)-N-((1-methoxynaphthalen-2-yl)methyl)piperidine-3-carboxamide hydrochloride.
(4S)—N-(2-chloro-3-methylbenzyl)-N-cyclopropyl-4-(4-(2-(2,6-dichloro-4-methylphenoxy)ethoxy)benzamido)piperidine-3-carboxamide hydrochloride [Non polar isomer].
(4S)—N-(2-chloro-3-methylbenzyl)-N-cyclopropyl-4-(4-(2-(2,6-dichloro-4-methylphenoxy)ethoxy)benzamido)piperidine-3-carboxamide hydrochloride

The compounds of the present invention may be prepared using the methods described below, together with conventional techniques known to those skilled in the art of organic synthesis, or variations thereon as appreciated by those skilled in the art. Referred methods include, but are not limited to those described below, where all symbols are as defined earlier.

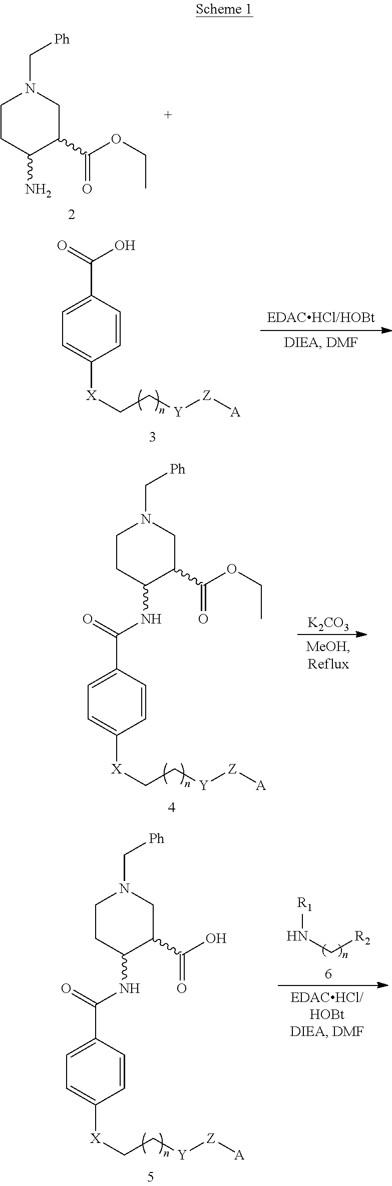

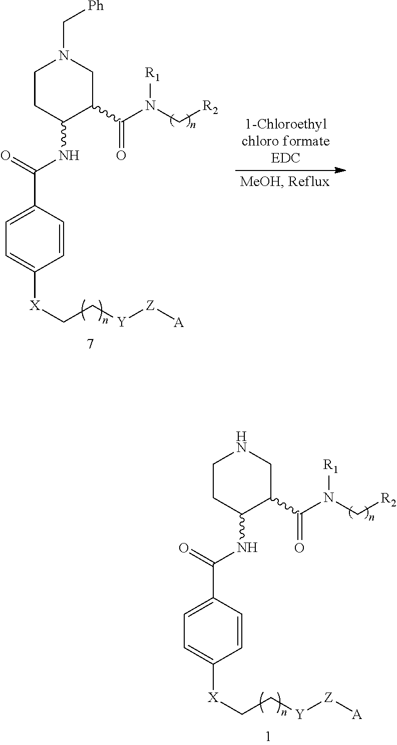

4-Benzamido-1-benzylpiperidine derivative of formula (4) where all symbols are defined earlier, may be synthesized by reacting amine derivative (2) where all symbols are as described earlier with benzoic acid derivative (3) using carboxyl group activating agents such as EDAC.HCl, dicyclohexyl carbodiimide and the like in the presence of an additive HOBT and base like triethyl amine or DIEA in solvent(s) like DMF or DCM at temperature 0-25° C. Hydrolysis of an ester group in 4-benzamido-1-benzylpiperidine derivative (4) by treating with base like lithium hydroxide, sodium hydroxide, potassium carbonate and like in protic solvent like methanol or ethanol at temperature 25-80° C. afford piperidine-3-carboxylic acid derivative (5). 3-Carboxamido piperidine (7) may be synthesized by reacting piperidine-3-carboxylic acid (5) with amine derivative (6) where all symbols are as described earlier, by using similar procedure described for preparation of 4-benzamido-1-benzylpiperidine derivative (4). Debenzylation of piperidine-3-carboxamide derivative (7) using various debenzylating agent like Pd/C—H₂(g), HCOOH—HCOONH₄, or 1-chloroethyl to chloroformate and like in a solvent like THF, methanol, DCM, EDC and mixture thereof at temperature 25-80° C. give piperidine derivative (1).

The amine derivative of formula [2] where all the symbols defined earlier can be synthesized as per the procedure mentioned in WO2006066747.

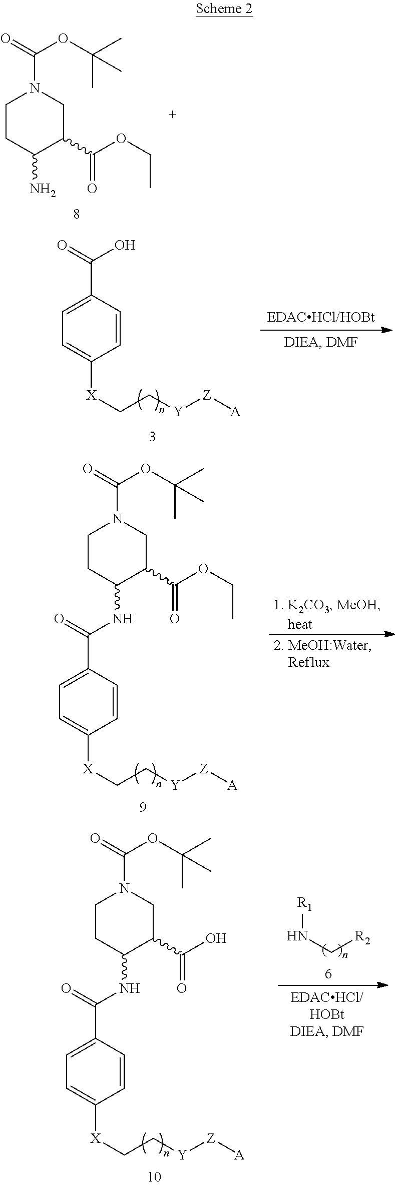

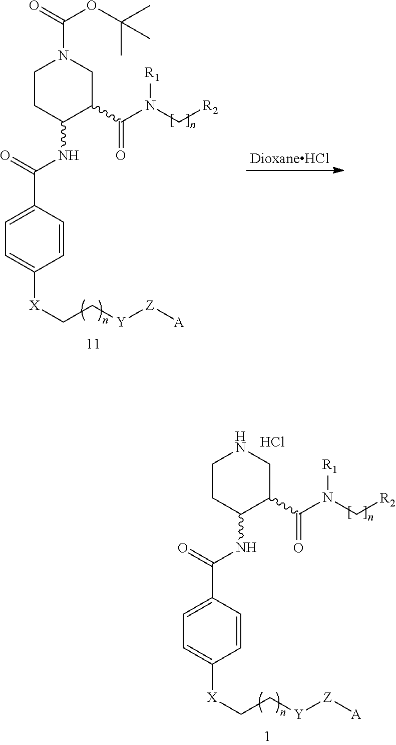

Reaction between (4S) or (4R)-1-tert-butyl 3-ethyl 4-aminopiperidine-1,3-dicarboxylate (8) where all symbols are as described earlier with benzoic acid derivative (3) using carboxyl group activating agents such as EDAC.HCl, dicyclohexyl carbodiimide and the like in the presence of an additive HOBT and base like triethyl amine or DIEA in solvent(s) like DMF or DCM at temperature 0-25° C. afforded 4(S) or 4(R) benzamide piperidine (9) as a mixture of cis and trans diastereomers. Epimerization in the presence of anhydrous K₂CO₃ in dry methanol or NaOEt in EtOH followed by hydrolysis of an ester group in 4(S) or 4(R) benzamide piperidine (9) by treating with base like lithium hydroxide, sodium hydroxide, potassium carbonate and like in protic solvent like methanol or ethanol at temperature 25-80° C. afford 4(S) or 4(R) piperidine-3-carboxylic acid derivative (10) as a mixture of cis and trans diastereomers. 3-Carboxamido piperidine (11) may be synthesized by reacting piperidine-3-carboxylic acid (10) with an appropriate amine derivative (6) where all symbols are as described earlier, by using similar procedure described for preparation of 4(S) or 4(R) benzamido piperidine (9). Both the diastereomers of piperidine (11) may be separated through column chromatography using known stationary phase like silica gel, alumina and by using mobile phase like EtOAc, hexane, chloroform, methanol and like or mixture thereof. Deprotection of BOC group in piperidine (11) by using various BOC deprotecting groups like TFA, dioxane.HCl, methanolic HCl and like at temperature 0-25° C. give piperidine derivative (1).

4(R) or (4S)-1-tert-butyl 3-ethyl 4-aminopiperidine-1,3-dicarboxylate (8) can be synthesised by following the procedure reported in WO0202525.

The benzoic acid derivative [3] where all the symbols defined earlier can be synthesized by variety of methods known to those skilled in the art following procedure set forth in reference such as Fieser and Fieser's Reagents for Organic Synthesis; Wiley & Sons: New York, Volumes 1-21; R. C. LaRock, Comprehensive Organic Transformations, 2.sup.nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-1 1; and Organic Reactions, Wiley & Sons: New York, 1991, Volumes 1-40, to name some of the known literature processes.

It will be appreciated that in any of the above mentioned reactions any reactive group in the substrate molecule may be protected, according to conventional chemical practice. Suitable protecting groups in any of the above mentioned reactions are those used conventionally in the art. The methods of formation and removal of such protecting groups are those conventional methods appropriate to the molecule being protected. T. W. Greene and P. G. M. Wuts “Protective groups in Organic Synthesis”, John Wiley & Sons, Inc, 1999, 3^{rd} Ed., 201-245 along with references therein gives such conventional methods and are incorporated herein as references.

The novel compounds of the present invention can be formulated into suitable pharmaceutically acceptable compositions by combining with suitable excipients by techniques and processes and concentrations as are well known.

The compounds of formula (1) or pharmaceutical compositions containing them are useful as Renin inhibitors suitable for humans and other warm blooded animals, and may be administered either by oral, topical or parenteral administration.

The pharmaceutically acceptable salts forming a part of this invention may be prepared by treating the compound of formula (1) with suitable acids in suitable solvents by processes known in the art.

The invention is further exemplified by the following examples below, which provides one of the several preferred embodiments of the present invention. These examples are provided merely as representative embodiments and should not be construed to limit the scope of the invention in any way.

### Preparation:

The Acid building blocks were synthesized by the process described beneath.

| Compound | Name |
|---|---|
| Acid 1 | 4-(2-(2,6-Dichloro-4-methylphenoxy)ethoxy)benzoic acid |
| Acid 2 | 4-(2-(2,6-Dichlorophenoxy)ethoxy)benzoic acid |
| Acid 3 | 4-(2-(2,6-Difluorophenoxy)ethoxy)benzoic acid |
| Acid 4 | 4-(2-((2-Methoxybenzyl)oxy)ethoxy)benzoic acid |
| Acid 5 | 4-(3-((2-Methoxybenzyl)oxy)propoxy)benzoic acid |

### Acid 1: 4-(2-(2,6-Dichloro-4-methylphenoxy)ethoxy)benzoic acid

### Step 1: 2-(2,6-Dichloro-4-methyl-phenoxy)-ethanol

2,6-Dichloro-4-methyl phenol (1 eq), ethylene carbonate (1.5 eq) and piperidine (0.1 eq) were combined and heated at 140° C. for 6 h to afford the title compound as brown oil.

### Step 2: 2-(2,6-Dichloro-4-methylphenoxy)ethyl methanesulfonate

2-(2,6-Dichloro-4-methyl-phenoxy)-ethanol (1 eq) obtained from step 1 above, and triethyl amine (3 eq) were taken up in 5 v of DCM. To this, then added methane sulfonyl chloride (1.2 eq) at 0-10° C. Mixture was allowed the attain room temperature and stirred for 4-6 h. Mixture was diluted with water and compound was extracted with DCM. Combined organic layer was washed with brine and dried over sodium sulfate. Filtration and concentration of filtrate in vacuo afforded a brown solid compound.

### Step 3: Methyl 4-(2-(2,6-dichloro-4-methylphenoxy)ethoxy)benzoate

2-(2,6-Dichloro-4-methylphenoxy)ethyl methanesulfonate (1 eq) obtained from step 2 above, and 4-hydroxy methyl benzoate (1 eq) were taken up in 5 v of DMF. To this anhydrous potassium carbonate (3 eq) was added and mixture of heated to 80-100° C. for 4-6 h. Mixture was diluted with water. Solid product obtained was filtered washed with water, dried under in vacuo afforded title compound as off white solid.

### Step 4: Acid 1

Methyl 4-(2-(2,6-dichloro-4-methylphenoxy)ethoxy)benzoate (1 eq) obtained from step 3 above, and lithium hydroxide (3 eq) were taken up in a mixture of THF:Water (5 v, 50%). Mixture was stirred for 4-6 h at room temperature. Mixture was diluted with water, acidification with dil. HCl afforded title compound as white solid.

### Acid 2: 4-(2-(2,6-Dichlorophenoxy)ethoxy)benzoic acid

Prepared similar to the procedure described in Acid 1 but using instead 2,6-dichloro phenol as a starting material.

### Acid 3: 4-(2-(2,6-Difluorophenoxy)ethoxy)benzoic acid

Prepared similar to the procedure described in Acid 1 but using instead 2,6-difluorophenol as a starting material.

### Acid 4: 4-(2-((2-Methoxybenzyl)oxy)ethoxy)benzoic acid

### Step 1: 2-((2-Methoxybenzyl)oxy)ethanol

Sodium hydride (2.2 eq) was added to a stirred solution of (2-methoxyphenyl)methanol (1 eq) in dry THF (10 v) at 0-5° C. and then, 2-bromoethanol (1.2 eq) was added. Mixture was stirred for 6 h at 10-25° C. Mixture was diluted with water, compound was extracted with EtOAc (20 v×2). Organic volatiles were removed in vacuo afforded title compound as colorless oil.

### Step 2: 2-((2-Methoxybenzyl)oxy)ethyl methanesulfonate

Prepared similar to the procedure described in Step 2 of Acid 1 but using instead 2-((2-methoxybenzyl)oxy)ethanol as a starting material.

### Step 3: Methyl 4-(2-((2-methoxybenzyl)oxy)ethoxy)benzoate

2-((2-Methoxybenzyl)oxy)ethyl methanesulfonate (1 eq) obtained from step 2 above, and 4-hydroxy methyl benzoate (1 eq) were taken up in 5 v of DMF. To this anhydrous potassium carbonate (3 eq) was added and mixture was heated to 80-100° C. for 4-6 h. Mixture was diluted with water. Solid product obtained was filtered washed with water, dried in vacuo afforded title compound as off white solid.

### Step 4: Acid 4

Methyl 4-(2-((2-methoxybenzyl)oxy)ethoxy)benzoate (1 eq) obtained from step 3 above, and lithium hydroxide (3 eq) were taken up in a mixture of THF:Water (5 v, 50%). Mixture was stirred for 4-6 h at room temperature. Mixture was diluted with water, acidification with dil. HCl afforded title compound as white solid.

### Acid 5: 4-(3-((2-Methoxybenzyl)oxy)propoxy)benzoic acid

Prepared similar to the procedure described in Acid 4 but using instead 3-bromo-1-propanol as a starting material.
The amine building blocks were synthesized by the process described beneath.

| | Name |
|---|---|
| Amine 1 | Piperidine |
| Amine 2 | N-(2,3-Dichlorobenzyl)cyclopropanamine |
| Amine 3 | N-(3-(3-Methoxypropoxy)benzyl)cyclopropanamine |
| Amine 4 | N-(3,5-Dimethylbenzyl)cyclopropanamine |
| Amine 5 | N-(2,4-Dichlorobenzyl)cyclopropanamine |
| Amine 6 | N-(3,4-Dimethoxybenzyl)cyclopropanamine |
| Amine 7 | N-(Naphthalen-2-ylmethyl)cyclopropanamine |
| Amine 8 | 3-(Methoxymethyl)piperidine hydrochloride |
| Amine 9 | N-(2-Chloro benzyl)cyclopropanamine |
| Amine 10 | N-(2-Fluoro benzyl)cyclopropanamine |
| Amine 11 | N-(2,3-Dimethoxybenzyl)cyclopropanamine |
| Amine 12 | N-((2,3-Dihydrobenzo[b][1,4]dioxin- |
| | 6-yl)methyl)cyclopropanamine |
| Amine 13 | N-(2-Chloro-4-fluorobenzyl)cyclopropanamine |
| Amine 14 | N-(2-Methoxy benzyl)cyclopropanamine |
| Amine 15 | N-(2-Chloro-4-(3-methoxypropyl)benzyl)cyclopropanamine |
| Amine 16 | N-(Benzo[d][1,3]dioxol-5-ylmethyl)cyclopropanamine |
| Amine 17 | N-(2-Chloro-3-methylbenzyl)cyclopropanamine |
| Amine 18 | N-(2,3-Dimethylbenzyl)cyclopropanamine |
| Amine 19 | N-(3,4-Dimethylbenzyl)cyclopropanamine |
| Amine 20 | N-(3-Methoxy-2-methylbenzyl)cyclopropanamine |
| Amine 21 | N((2,3-Dihydrobenzo[b][1,4]dioxin- |
| | 5-yl)methyl)cyclopropanamine |
| Amine 22 | N-((1-Methoxynaphthalen-2-yl)methyl)cyclopropanamine |

### Amine 2: N-(2,3-Dichlorobenzyl)cyclopropanamine

A mixture of 2,3-dichloro benzaldehyde (1 eq), cyclopropyl amine (1.2 eq) and sodium bicarbonate (1.5 eq) were heated at refluxed in methanol (10 v) for 1 h. The reaction mixture was then cooled in ice and sodium borohydride (1.2 eq) was introduced portion wise. After complete addition mixture was allowed to warm to RT and stirred for 3 h. The volatiles were then removed in vacuo and the resulting residue was partitioned between water and DCM. The organic layer was separated, washed with water, dried over sodium sulfate and filtered. Concentration of the organic layer in vacuo afforded the title compound as light yellow oil. Purification of crude product thus obtained by the way of chromatography using (Silica, Hexane to 40% EtOAc in Hexane) afforded title compound as light yellow oil.

### Amine 3: N-(3-(3-Methoxypropoxy)benzyl)cyclopropanamine

Prepared similar to the procedure described in Amine 2 but using instead 3-(3-methoxypropoxy)benzaldehyde as a starting material.

### Amine 4: N-(3,5-Dimethylbenzyl)cyclopropanamine

Prepared similar to the procedure described in Amine 2 but using instead 3,5-dimethyl benzaldehyde as a starting material.

### Amine 5: N-(2,4-Dichlorobenzyl)cyclopropanamine

Prepared similar to the procedure described in Amine 2 but using instead 2,4-dichloro benzaldehyde as a starting material.

### Amine 6: N-(3,4-Dimethoxybenzyl)cyclopropanamine

Prepared similar to the procedure described in Amine 2 but using instead 3,4-dimethoxy benzaldehyde as a starting material.

### Amine 7: N-(Naphthalen-2-ylmethyl)cyclopropanamine

Prepared similar to the procedure described in Amine 2 but using instead 2-napthaldehyde as a starting material.

### Amine 8: 3-Methoxymethyl-piperidine hydrochloride

### Step 1: 3-Methanesulfonyloxymethyl-piperidine-1-carboxylic acid tert-butyl ester

N-Boc-3-hydroymethyl piperidine (1 eq), triethylamine (2 eq) were taken in DCM (10 v). Methane sulfonyl chloride (1.2 eq) was added to the mixture at 0° C. Mixture was allowed to stirred at 10° C. for 4 h. Reaction mixture was quenched in water. Organic layer was separated washed with brine, dried over sodium sulfate, filtered, and evaporated in vacuo to get title compound as semi solid.

### Step 2.: 3-Methoxymethyl-piperidine-1-carboxylic acid tert-butyl ester

Sodium metal (3 eq) was dissolved in dry methanol (10 v) and 3-methanesulfonyloxy methyl-piperidine-1-carboxylic acid tert-butyl ester (1 eq) was added to this stirred solution at 10° C. Mixture was refluxed form 3 h. Mixture was quenched in water. Product was extracted by EtOAc. Organic layer was washed with water, brine, dried over sodium sulfate, filtered and evaporated in vacuo to afford title compound as liquid.

### Step 3.: Amine 8

Dioxane.HCl (10%) was added to 3-methoxymethyl-piperidine-1-carboxylic acid tert-butyl ester (1 eq) and mixture was stirred for 1 h at 0-5° C. Organic volatiles were removed under reduced pressure to afford title compound as yellow solid.

### Amine 9: N-(2-Chloro benzyl)cyclopropanamine

Prepared similar to the procedure described in Amine 2 but using instead 2-chloro benaldehyde as a starting material.

### Amine 10: N-(2-Fluoro benzyl)cyclopropanamine

Prepared similar to the procedure described in Amine 2 but using instead 2-fluoro benaldehyde as a starting material.

### Amine 11: N-(2,3-Dimethoxybenzyl)cyclopropanamine

Prepared similar to the procedure described in Amine 2 but using instead 2,3-dimethoxy benaldehyde as a starting material.

### Amine 12: N-((2,3-Dihydrobenzo[b][1,4]dioxin-6-yl)methyl)cyclopropanatnine

Prepared similar to the procedure described in Amine 2 but using instead 2,3-dihydrobenzo[b][1,4]dioxine-6-carbaldehyde as a starting material.

### Amine 13: N-(2-Chloro-4-fluorobenzyl)cyclopropanamine

Prepared similar to the procedure described in Amine 2 but using instead 2-chloro-4-fluoro benaldehyde as a starting material.

### Amine 14: N-(2-Methoxy benzyl)cyclopropanamine

Prepared similar to the procedure described in Amine 2 but using instead 2-methoxy benaldehyde as a starting material.

### Amine 15: N-(2-Chloro-4-(3-methoxypropyl)benzyl)cyclopropanamine

Prepared similar to the procedure described in Amine 2 but using instead 2-chloro-4-(3-methoxypropyl)benzaldehyde a starting material.

### Amine 16: N-(Benzo[d][1,3]dioxol-5-ylmethyl)cyclopropanamine

Prepared similar to the procedure described in Amine 2 but using instead benzo[d][1,3]dioxole-5-carbaldehyde as a starting material.

### Amine 17: N-(2-Chloro-3-methylbenzyl)cyclopropanamine

### Step 1. 2-Chloro-N-cyclopropyl-3-methylbenzamide

To a solution of 2-chloro-3-methyl benzoic acid (1 eq) in DMF (5 v), was added HOBT (1.5 eq). To this reaction mixture, was added EDAC.HCl (1.2 eq), cyclopropylamine (1.2 eq) and DIEA (1.5 eq) under N₂ at 0-5° C. The resulting reaction mixture was stirred at 25° C. for 16 h. Mixture was quenched in water, solid obtained was filtered, dried in vacuo to afforded title compound as off white solid.

### Step 2: Amine 17

2-Chloro-N-cyclopropyl-3-methylbenzamide (1 eq) obtained from step 1 above was dissolved in dry THF and boron dimethyl sulfide was added dropwise at 60° C. After completion of reaction organic volatiles were removed in vacuo. Mixture was decomposed by adding 50% HCl solution. Mixture was basified by adding ammonia solution. Compound was extracted by using EtOAc (20 V×2). Combined organic layer was washed with water, brine, dried over sodium sulfate and removed in vacuo afforded oily compound. Crude product thus was purified by the way of flash chromatography using silica gel G mobile phase Hexane to 50% ethyl acetate in hexane. Title compound obtained as light yellow liquid.

### Amine 18: N-(2,4-Dimethylbenzyl)cyclopropanamine

Prepared similar to the procedure described in Amine 17 but using instead 2,4-dimethyl benzoic acid as a starting material.

### Amine 19: N-(3,4-Dimethylbenzyl)cyclopropanamine

Prepared similar to the procedure described in Amine 17 but using instead 3,4-dimethyl benzoic acid as a starting material.

### Amine 20: N-(3-Methoxy-2-methylbenzyl)cyclopropanamine

Prepared similar to the procedure described in Amine 17 but using instead 3-methoxy-2-methyl benzoic acid as a starting material.

### Amine 21: N-β2,3-Dihydrobenzo[b][1,4]dioxin-5-yl)methyl)cyclopropanamine

Prepared similar to the procedure described in Amine 17 but using instead 2,3-dihydrobenzo[b][1,4]dioxine-5-carboxylic acid as a starting material.

### Amine 22: N-((1-Methoxynaphthalen-2-yl)methypcyclopropanamine Prepared similar to the procedure described in Amine 17 but using instead 1-methoxy-2-napthoic acid a starting material

HPLC Conditions:

a) HPLC Column: YMC J sphere C 18(150*4.6 mm) 4u
Mobile phase: 0.05 TFA: ACN gradient.
Flow rate: 1.0 mL/min.
Wave length: UV at 220 nm.

### b) HPLC Column: ODS C 18(150*4.6 mm) 4u

Mobile phase: 0.05 TFA: ACN gradient.
Flow rate: 1.0 ml/min.
Wave length: UV at 220 nm.

### c) HPLC Column: YMC AQ (150*4.6 mm) 4u

Mobile phase: 0.05 TFA: ACN gradient.
Flow rate: 1.0 ml/min.
Wave length: UV at 220 nm.
   d) HPLC Column: UMC-J′ sphere, ODS-H80 (150*4.6 mm) 4u
Mobile phase: 0.05 TFA: ACN gradient.
Flow rate: 1.0 rill/min.
Wave length: UV at 220 nm.

### Example 1

### Scheme 1

### Preparation of N-cyclopropyl-N-(2,3-dichlorobenzyl)-4-(4-(2-(2,6-dichlorophenoxy)ethoxy)benzamido) piperidine-3-carboxamide

### Step 1: Preparation of ethyl 1-benzyl-4-(4-(2-(2,6-dichlorophenoxy)ethoxy)benzamido) piperidine-3-carboxylate

To a solution of 4-(2-(2,6-dichlorophenoxy)ethoxy)benzoic acid [6.15 g, 18.81 mmol] in 60 mL DMF, was added HOBT [4.48 g, 33.20 mmol]. To this reaction mixture, was added EDAC.HCl [5.52 g, 28.77 mmol], ethyl 4-amino-1-benzylpiperidine-3-carboxylate [5.8 g, 22.1 mmol] and DIEA [8.56 g, 66.41 mmol] under N₂ at 0-5° C. The resulting reaction mixture was stirred at 25° C. for 16 h. Mixture was diluted with water. The aqueous layer was extracted with EtOAc. The organic layer was washed with brine, dried over sodium sulfate and concentrated in vacuo to afford off white solid compound. Purification of crude product was thus obtained by the way of column chromatogaphy (Silica gel G, Mesh size 230-400, hexane to 30% EtOAc in hexane) to get solid (5 g, 72%, m.p.: 144-145° C.). The title compound was characterized by spectral analysis. ESI-MS: 571.05 (M+H)⁺

### Step 2: 1-Benzyl-4-(4-(2-(2,6-dichlorophenoxy)ethoxy)benzamido)piperidine-3-carboxylic acid

To a solution of ethyl 1-benzyl-4-(4-(2-(2,6-dichlorophenoxy)ethoxy)benzamido) piperidine-3-carboxylate [5.0 g, 8.75 mmol] in methanol:water (4:1,(v/v)), potassium carbonate [3.62 g, 26.2 mmol] was added and mixture was refluxed for 16 h. Mixture was cooled to room temperature and quenched in water. Acidified the mixture to pH ˜2 by using 5% aq HCl. Solid obtained was filtered, washed with water and dried. The title compound was isolated as white solid (3.4 g, 72%, m.p. 204-207° C.) and characterized by spectral analysis. ESI-MS: 543.03 (M+H)⁺

### Step 3: 1-Benzyl-N-cyclopropyl-N-(2,3-dichlorobenzyl)-4-(4-(2-(2,6-dichlorophenoxy)ethoxy)benzamido)piperidine-3-carboxamide

To a solution of 1-benzyl-4-(4-(2-(2,6-dichlorophenoxy)ethoxy)benzamido)piperidine-3-carboxylic
acid [3 g, 5.52 mmol] in 50 ml of dry DMF, was added HOBT [1.11 g, 8.28 mmol]. To this, reaction mixture was added, EDAC. HCl [.1.37 g, 7.18 mmol], N-(2,3-dichlorobenzyl)cyclopropanamine [1.19 g, 5.52 mmol] and DIEA [2.1 g, 16.57 mmol] under N₂ at 0-5° C. The resulting reaction mixture was stirred at 25° C. for 16 h. Mixture was diluted with water. The aqueous layer was extracted with EtOAc. The organic layer was washed with brine, dried over sodium sulfate and concentrated in vacuo to afford off white solid compound. Purification of crude product was thus obtained by the way of column chromatogaphy (SiO₂, hexane to 50% EtOAc in hexane) to get low melting solid (1.65 g, 40%). The title compound was characterized by spectral analysis. ESI-MS: 742 (M+H)⁺

### Step 4: N-cyclopropyl-N-(2,3-dichlorobenzyl)-4-(4-(2-(2,6-dichlorophenoxy)ethoxy)benzamido) piperidine-3-carboxamide

To a solution of 1-benzyl-N-cyclopropyl-N-(2,3-dichlorobenzyl)-4-(4-(2-(2,6-dichlorophenoxy)ethoxy)benzamido)piperidine-3-carboxamide [1.65 g, 2.22 mmol] obtained from step 3 and 1-chloroethyl chloroformate [0.636 g, 4.45 mmol] in EDC, was added sodium bicarbonate [0.56 g, 6.68 mmol]. Reaction mixture was stirred at 25° C. for 16 h. Organic volatiles were removed under reduced pressure. Methanol was added to this gummy mass and refluxed for 3 h. Mixture was quenched in water. The aqueous layer was extracted with EtOAc. The organic layer was washed with brine, dried over sodium sulfate and concentrated in vacuo to afford off white solid compound. Purification of crude product was thus obtained by the way of column chromatogaphy (SiO₂, 10% MeOH in CHCl₃) to get low melting solid (0.650 g, 45%, m.p. 66-68° C.). The title compound was characterized by spectral analysis. ESI-MS: 651.8 (M+H)⁺. ^{b}HPLC tᵣₑₜ: 14.99 min.
The following compounds were prepared by following the general process described in Example 1.

### Example 2

### 4-(2-(2,6-Dichlorophenoxy)ethoxy)-N-(3-(piperidine-1-carbonyl)piperidin-4-yl)benzamide

Prepared similar to the procedure described in Example 1 but using instead Acid 2 and Amine 1 as a starting material. The title compound was obtained as thick liquid ESI-MS: M⁺=520; ^{a}HPLC tᵣₑₜ: 14.99 min.

### Example 3

### N-Cyclopropyl-4-(4-(2-(2,6-dichlorophenoxy)ethoxy)benzamido)-N-(3-(3-methoxy propoxy)benzyl)piperidine-3-carboxamide

Prepared similar to the procedure described in Example 1 but using instead Acid 2 and Amine 3 as a starting material. The title compound was obtained as thick liquid ESI-MS: M⁺=670; ^{a}HPLC tᵣₑₜ: 16.45 min.

### Example 4

### N-Cyclopropyl-4-(4-(2-(2,6-dichlorophenoxy)ethoxy)benzamido)-N-(3,5-dimethylbenzyl)piperidine-3-carboxamide

Prepared similar to the procedure described in Example 1 but using instead Acid 2 and Amine 4 as a starting material. The title compound was obtained as a mixture of two diastereomers. ESI-MS: M⁺=610; ^{a}HPLC tᵣₑₜ: 18.01 & 18.51 min.

### Example 5

### N-Cyclopropyl-4-(4-(2-(2,6-dichloro-4-methylphenoxy)ethoxy)benzamido)-N-(2,4-dichlorobenzyl)piperidine-3-carboxamide

Prepared similar to the procedure described in Example 1 but using instead Acid 1 and Amine 5 as a starting material. The title compound was obtained as a mixture of two diastereomers. ESI-MS: M⁺=666; ^{a}HPLC tᵣₑₜ: 19.51 & 19.87 min.

### Example 6

### N-Cyclopropyl-N-(2,4-dichlorobenzyl)-4-(4-(2-(2,6-dichlorophenoxy)ethoxy)benzamido) piperidine-3-carboxamide

Prepared similar to the procedure described in Example 1 but using instead Acid 2 and Amine 5 as a starting material. The title compound was obtained as thick liquid. ESI-MS: M⁺=651; ^{a}HPLC tᵣₑₜ: 18.66 min.

### Example 7

### N-Cyclopropyl-4-(4-(2-(2,6-dichlorophenoxy)ethoxy)benzamido)-N-(3,4-dimethoxybenzyl)piperidine-3-carboxamide

Prepared similar to the procedure described in Example 1 but using instead Acid 2 and Amine 6 as a starting material. The title compound was obtained as thick liquid. ESI-MS: M⁺=642; ^{a}HPLC tᵣₑₜ: 16.31 min.

### Example 8

### N-Cyclopropyl-4-(4-(2-(2,6-dichloro-4-methylphenoxy)ethoxy)benzamido)-N-(3,4-dimethoxybenzyl)piperidine-3-carboxamide [Polar isomer]

Prepared similar to the procedure described in Example 1 but using instead Acid 1 and Amine 6 as a starting material. The title compound was obtained as thick liquid. ESI-MS: M⁺=656; ^{a}HPLC tᵣₑₜ: 17.09 min.

### Example 9

### N-Cyclopropyl-4-(4-(2-(2,6-dichloro-4-methylphenoxy)ethoxy)benzam ido)-N-(3,4-dimethoxybenzyl)piperidine-3-carboxamide [Non polar isomer]

Prepared similar to the procedure described in Example 1 but using instead Acid 1 and Amine 6 as a starting material. The title compound was obtained as thick liquid. ESI-MS: M⁺=656; ^{a}HPLC tᵣₑₜ: 16.80 min.

### Example 10

### N-Cyclopropyl-4-(4-(2-(2,6-dichloro-4-methylphenoxy)ethoxy)benzamido)-N-(3,5-dimethylbenzyl)piperidine-3-carboxamide

Prepared similar to the procedure described in Example 1 but using instead Acid 1 and Amine 4 as a starting material. The title compound was obtained as a mixture of diastereomers. ESI-MS: M⁺=624; ^{a}HPLC tᵣₑₜ: 19.03 & 19.30 min.

### Example 11

### N-Cyclopropyl-4-(4-(2-(2,6-dichlorophenoxy)ethoxy)benzamido)-N-(naphthalen-2-yl methyl)piperidine-3-carboxamide

Prepared similar to the procedure described in Example 1 but using instead Acid 2 and Amine 7 as a starting material. The title compound was obtained as a thick liquid. ESI-MS: M⁺=632; ^{a}HPLC tᵣₑₜ: 18.18 min.

### Example 12

### N-Cyclopropyl-4-(4-(2-(2,6-dichloro-4-methylphenoxy)ethoxy)benzamido)-N-(naphthalen-2-ylmethyl)piperidine-3-carboxamide

Prepared similar to the procedure described in Example 1 but using instead Acid 1 and Amine 7 as a starting material. The title compound was obtained as a thick liquid. ESI-MS: M⁺=646; ^{a}HPLC tᵣₑₜ: 17.42 min.

### Example 13

### 4-(2-(2,6-Dichloro-4-methylphenoxy)ethoxy)-N-(3-(3-(methoxymethyl)piperidine-1-carbonyl)piperidin-4-yl)benzamide

Prepared similar to the procedure described in Example 1 but using instead Acid 1 and Amine 8 as a starting material. The title compound was obtained as a thick liquid. ESI-MS: M⁺=578.10; ^{a}HPLC tᵣₑₜ: 17.89 min.

### Example 14 & 15

### Preparation of (4S)—N-cyclopropyl-4-(4-(2-(2,6-dichloro-4-methylphenoxy)ethoxy)benzamido)-N-(2,3-dimethoxybenzyl)piperidine-3-carboxamide hydrochloride. [Polar & non polar isomer]

### Step 1: (4S)-1-tert-butyl 3-ethyl 4-(4-(2-(2,6-dichloro-4-methylphenoxy)ethoxy)benzamido)-1,3-dicarboxylate

To a solution of 4-(2-(2,6-dichloro-4-methyl phenoxy)ethoxy)benzoic acid [2.5 g, 7.35 mmol] in 60 mL DMF, was added HOBT [1.5 g, 11.02 mmol]. To this reaction mixture, was added EDAC.HCl [1.7 g, 8.82 mmol], (4S)-1-tert-butyl 3-ethyl-4-amino piperidine-1,3-dicarboxylate [2.0 g, 7.35 mmol] and DIEA [2.84 g, 22.05 mmol] under N₂ at 0-5° C. The resulting reaction mixture was stirred at 25° C. for 16 h. Mixture was diluted with water. The aqueous layer was extracted with EtOAc. The organic layer was washed with brine, dried over sodium sulfate and concentrated in vacuo to afford thick liquid compound as a mixture of diastereomers. Purification of crude product was thus obtained by the way of column chromatography (SiO₂, hexane to 30% EtOAc in hexane) to get oily compound (2.85 g, 65%). The title compound was isolated as a mixture of diastereomers and characterized by spectral analysis ESI-MS: 617; ^{a}HPLC tᵣₑₜ: 23.97 & 24.80 min.

### Step 2: (4S)-1-(tert-butoxycarbonyl)-4-(4-(2-(2,6-dichloro-4-methylphenoxy)ethoxy)benzamido)piperidine-3-carboxylic acid

To a solution of (4S)-1-tert-butyl 3-ethyl 4-(4-(2-(2,6-dichloro-4-methylphenoxy)ethoxy)benzamido)piperidine-1,3-dicarboxylate [2.8 g, 4.7 mmol] in methanol (5 v) and molecular sieves [4A°] was refluxed for 2 h and anhydrous potassium carbonate [1.91 g, 14 mmol] was added and mixture was heated for another 5 h. Mixture was filtered through hyflow, methanol:water (4:1,(v/v)), potassium carbonate [1.91 g, 14 mmol] was added and mixture was refluxed for 16 h. Mixture was cooled to room temperature and quenched in water. Acidified the mixture to pH ˜4 by using 5% aq HCl. Solid obtained was filtered, washed with water and dried.
The title compound was isolated as a mixture of diastereomers as white solid (2.2 g, 82.70%) and characterized by spectral analysis. ESI-MS: 567 (M)+; ^{a}HPLC tᵣₑₜ: 21.37 & 21.42 min.

### Step 3: (4S)-tert-butyl 3-(cyclopropyl(2,3-dimethoxybenzyl)carbamoyl)-4-(4-(2-(2,6-dichloro-4-methylphenoxy)ethoxy)benzamido)piperidine-1-carboxylate

To a solution of (4S)-1-(tert-butoxycarbonyl)-4-(4-(2-(2,6-dichloro-4-methylphenoxy)ethoxy)benzamido)piperidine-3-carboxylic acid [0.8 g, 1.4 mmol] in 15 mL DMF, was added HOBT [0.28 g, 2.1 mmol]. To this reaction mixture, was added EDAC.HCl [0.32 g, 1.6 mmol], N-(2,3-dimethoxybenzyl)cyclopropanamine [0.29 g, 1.4 mmol] and DIEA [0.54 g, 4.2 mmol] under N₂ at 0-5° C. The resulting reaction mixture was stirred at 25° C. for 16 h. Mixture was diluted with water. The aqueous layer was extracted with EtOAc. The organic layer was washed with brine, dried over sodium sulfate and concentrated in vacuo to afford thick liquid compound as a mixture of diastereomers. Both the diastereomers were separated by the way of column chromatography (SiO₂, hexane to 50% EtOAc in hexane). Non polar isomer obtained (0.19 g, 18.11%) as thick liquid and characterized by spectral analysis. ESI-MS: 756 (M)⁺; aHPLC tᵣₑₜ 25.76 min. Polar isomer obtained (0.425 g, 40%) as a semisolid mass and characterized by spectral analysis ESI-MS: 756 (M)⁺, ^{a}HPLC tᵣₑₜ 24.31 min.

### Step 4: Example 14

To a solution of (4S)-tert-butyl 3-(cyclopropyl(2,3-dimethoxybenzyl)carbamoyl)-4-(4-(2-(2,6-dichloro-4-methylphenoxy)ethoxy)benzamido)piperidine-1-carboxylate (Non Polar isomer) (0.190 g, 0.25 mmol) in 10 v of DCM was added Dioxane. HCl (4M, 20 eq) and mixture was stirred at RT for 2 h. Removal of organic volatiles afforded a title compound as hydrochloride salt. The compound was characterized by spectral analysis. ESI-MS: 656 (M)⁺; ^{a}HPLC tᵣₑₜ: 18.19 min.

### Step 4: Example 15

To a solution of (4S)-tert-butyl 3-(cyclopropyl(2,3-dimethoxybenzyl)carbamoyl)-4-(4-(2-(2,6-dichloro-4-methylphenoxy)ethoxy)benzamido)piperidine-1-carboxylate (Polar isomer) (0.425 g, 0.56 mmol) in 10 v of DCM was added dioxane. HCl (4M, 20 eq) and mixture was stirred at RT for 2 h. Removal of organic volatiles afforded a title compound as hydrochloride salt. The compound was characterized by spectral analysis. ESI-MS: 656 (M)⁺; ^{a}HPLC tᵣₑₜ: 17.66 min.

The following compounds were prepared by following the general process described in Example 14 or in Example 15.

### Example 16

### (4S)—N-Cyclopropyl-N-(2,3-dimethylbenzyl)-4-(4-(2-((2-methoxybenzyl)oxy)ethoxy)benzamido)piperidine-3-carboxamide. [Polar isomer]

Prepared similar to the procedure described in Example 15 but using instead Acid 4 and Amine 18 as a starting material. The title compound was obtained as a thick liquid. ESI-MS: M⁺=586; ^{c}HPLC tᵣₑₜ: 16.73 min.

### Example 17

### (4S)—N-Cyclopropyl-N-(2,3-dimethylbenzyl)-4-(4-(2-((2-methoxybenzyl)oxy)ethoxy)benzamido)piperidine-3-carboxamide.[Non Polar isomer]

Prepared similar to the procedure described in Example 14 but using instead Acid 4 and Amine 18 as a starting material. The title compound was obtained as a thick liquid. ESI-MS: M⁺=586; ^{c}HPLC tᵣₑₜ: 17.05 min.

### Example 18

### (4S)—N-Cyclopropyl-4-(442-(2,6-dichloro-4-methylphenoxy)ethoxy)benzamido)-N-(3,4-dimethylbenzyl)piperidine-3-carboxamide hydrochloride. [Polar isomer]

Prepared similar to the procedure described in Example 15 but using instead Acid 1 and Amine 19 as a starting material. The title compound was obtained as a thick liquid. ESI-MS: M⁺=624; ^{a}HPLC tᵣₑₜ: 18.23 min.

### Example 19

### (4S)—N-Cyclopropyl-4-(4-(2-(2,6-dichloro-4-methylphenoxy)ethoxy)benzamido)-N-(2,3-dimethylbenzyl)piperidine-3-carboxamide hydrochloride [Nonpolar isomer]

Prepared similar to the procedure described in Example 14 but using instead Acid 1 and Amine 18 as a starting material. The title compound was obtained as a thick liquid. ESI-MS: M⁺=624; ^{a}HPLC tᵣₑₜ: 18.64 min.

### Example 20

### (4S)—N-Cyclopropyl-4-(4-(2-(2,6-dichloro-4-methylphenoxy)ethoxy)benzamido)-N-(2,3-dimethylbenzyl)piperidine-3-carboxamide hydrochloride. [Polar isomer]

Prepared similar to the procedure described in Example 15 but using instead Acid 1 and Amine 18 as a starting material. The title compound was obtained as a thick liquid. ESI-MS: M⁺=624; ^{a}HPLC tᵣₑₜ: 18.37 min.

### Example 21

### (4S)—N-Cyclopropyl-4-(4-(2-(2,6-dichloro-4-methylphenoxy)ethoxy)benzamido)-N-(3,4-dimethylbenzyl)piperidine-3-carboxamide hydrochloride [Non polar isomer]

Prepared similar to the procedure described in Example 14 but using instead Acid 1 and Amine 19 as a starting material. The title compound was obtained as a thick liquid. ESI-MS: M⁺=624; ^{a}HPLC tᵣₑₜ: 18.69 min.

### Example 22

### (4R)—N-Cyclopropyl-4-(4-(2-(2,6-dichloro-4-methylphenoxy)ethoxy)benzamido)-N-(3,4-dimethylbenzyl)piperidine-3-carboxamide hydrochloride [Polar isomer]

Prepared similar to the procedure described in Example 15 but using instead Acid 1, Amine 19 and (4R)-1-tert-butyl 3-ethyl-4-amino piperidine-1,3-dicarboxylate as a starting material. The title compound was obtained as a thick liquid. ESI-MS: M⁺=624; ^{a}HPLC tᵣₑₜ: 18.34 min.

### Example 23

### (4R)—N-Cyclopropyl-4-(4-(2-(2,6-dichloro-4-methylphenoxy)ethoxy)benzamido)-N-(3,4-dimethylbenzyl)piperidine-3-carboxamide hydrochloride [Non polar isomer]

Prepared similar to the procedure described in Example 14 but using instead Acid 1, Amine 19 and (4R)-1-tert-butyl 3-ethyl-4-amino piperidine-1,3-dicarboxylate as a starting material. The title compound was obtained as a thick liquid. ESI-MS: M⁺=624; ^{a}HPLC tᵣₑₜ: 18.72 min.

### Example 24

### (4R)—N-Cyclopropyl-4-(4-(2-(2,6-dichloro-4-methylphenoxy)ethoxy)benzamido)-N-(2,3-dimethylbenzyl)piperidine-3-carboxamide hydrochloride [Polar isomer]

Prepared similar to the procedure described in Example 15 but using instead Acid 1, Amine 18 and (4R)-1-tert-butyl 3-ethyl-4-amino piperidine-1,3-dicarboxylate as a starting material. The title compound was obtained as a thick liquid. ESI-MS: M⁺=624; ^{a}HPLC tᵣₑₜ: 18.37 min.

### Example 25

### (4R)—N-Cyclopropyl-4-(4-(2-(2,6-dichloro-4-methylphenoxy)ethoxy)benzamido)-N-(2,3-dimethylbenzyl)piperidine-3-carboxamide hydrochloride [Non polar isomer]

Prepared similar to the procedure described in Example 14 but using instead Acid 1, Amine 18 and (4R)-1-tert-butyl 3-ethyl-4-amino piperidine-1,3-dicarboxylate as a starting material. The title compound was obtained as a thick liquid. ESI-MS: M⁺=624; ^{a}HPLC tᵣₑₜ: 18.73 min.

### Example 26

### (4S)—N-Cyclopropyl-4-(4-(2-(2,6-dichloro-4-methylphenoxy)ethoxy)benzam ido)-N-(2,3-dimethylbenzyl)piperidine-3-carboxamide [Polar isomer]

### Step 1: (4S)—N-Cyclopropyl-4-(4-(2-(2,6-dichloro-4-methylphenoxy)ethoxy)benzamido)-N-(2,3-dimethylbenzyl)piperidine-3-carboxamide hydrochloride[Polar isomer]

Prepared similar to the procedure described in Example 15 but using instead Acid 1 and Amine 18 as a starting material. The title compound was obtained as a thick liquid.

### Step 2: Example 26

(4S)—N-Cyclopropyl-4-(4-(2-(2,6-dichloro-4-methylphenoxy)ethoxy)benzamido)-N-(2,3-dimethylbenzyl)piperidine-3-carboxamide hydrochloride (1 eq) obtained from step 1 was dissolved in DCM (5 v). Triethyl amine (1.5 eq) was added followed by (5 v) of water. Organic layer was separated and removed in vacuo to obtained low melting solid compound. The compound was characterized by spectral analysis. ESI-MS: M⁺=624; ^{a}HPLC tᵣₑₜ: 18.01 min.

### Example 27

### (4S)—N-Cyclopropyl-N-(2,3-dimethylbenzyl)-4-(4-(34(2-methoxybenzyl)oxy) propoxy)benzamido)piperidine-3-carboxamide hydrochloride [Non polar isomer]

Prepared similar to the procedure described in Example 14 but using instead Acid 5 and Amine 18 as a starting material. The title compound was obtained as a thick liquid. ESI-MS: M⁺=600; ^{a}HPLC tᵣₑₜ: 17.39 min.

### Example 28

### (4S)—N-Cyclopropyl-N-(2,3-dimethylbenzyl)-4-(4-(3-((2-methoxybenzyl)oxy)propoxy)benzamido)piperidine-3-carboxamide hydrochloride [Polar isomer]

Prepared similar to the procedure described in Example 15 but using instead Acid 5 and Amine 18 as a starting material. The title compound was obtained as a thick liquid. ESI-MS: M⁺=600; ^{a}HPLC tᵣₑₜ: 17.10 min.

### Example 29

### (4S)—N-Cyclopropyl-4-(4-(2-(2,6-dichloro-4-methylphenoxy)ethoxy)benzamido)-N-(2-fluorobenzyl)piperidine-3-carboxamide hydrochloride. [Polar isomer]

Prepared similar to the procedure described in Example 15 but using instead Acid 1 and Amine 10 as a starting material. The title compound was obtained as a thick liquid. ESI-MS: M⁺=614; ^{a}HPLC tᵣₑₜ: 18.04 min.

### Example 30

### (4S)—N-(2-Chlorobenzyl)-N-cyclopropyl-4-(4-(2-(2,6-dichloro-4-methylphenoxy)ethoxy)benzamido)piperidine-3-carboxamide hydrochloride [Polar isomer]

Prepared similar to the procedure described in Example 15 but using instead Acid 1 and Amine 9 as a starting material. The title compound was obtained as a thick liquid. ESI-MS: M⁺=632; ^{a}HPLC tᵣₑₜ: 18.68 min.

### Example 31

### (4S)—N-Cyclopropyl-4-(4-(2-(2,6-dichloro-4-methylphenoxy)ethoxy)benzam ido)-N-(2-methoxybenzyl)piperidine-3-carboxamide hydrochloride [Polar isomer]

Prepared similar to the procedure described in Example 15 but using instead Acid 1 and Amine 14 as a starting material. The title compound was obtained as a thick liquid. ESI-MS: M⁺=626; ^{a}HPLC tᵣₑₜ: 17.99 min.

### Example 32

### (4S)—N-Cyclopropyl-4-(4-(2-(2,6-dichloro-4-methylphenoxy)ethoxy)benzam ido)-N-(2-methoxybenzyl)piperidine-3-carboxamide hydrochloride [Non polar isomer]

Prepared similar to the procedure described in Example 14 but using instead Acid 1 and Amine 14 as a starting material. The title compound was obtained as a thick liquid. ESI-MS: M⁺=626; ^{a}HPLC tᵣₑₜ: 18.44 min.

### Example 33

### (4S)—N-Cyclopropyl-N-(2-methoxybenzyl)-4-(4-(3-((2-methoxybenzyl)oxy)propoxy)benzamido)piperidine-3-carboxamide hydrochloride [Polar isomer]

Prepared similar to the procedure described in Example 15 but using instead Acid 5 and Amine 14 as a starting material. The title compound was obtained as a thick liquid. ESI-MS: M⁺=602; ^{a}HPLC tᵣₑₜ: 16.94 min.

### Example 34

### (4S)—N-Cyclopropyl-N-(2-methoxybenzyl)-4-(4-(3-((2-methoxybenzyl)oxy)propoxy)benzamido)piperidine-3-carboxamide hydrochloride [Non polar isomer]

Prepared similar to the procedure described in Example 14 but using instead Acid 5 and Amine 14 as a starting material. The title compound was obtained as a thick liquid. ESI-MS: M⁺=602; ^{a}HPLC fᵣₑₜ: 17.02 min.

### Example 35

### (4S)—N-Cyclopropyl-4-(4-(2-(2,6-dichloro-4-methylphenoxy)ethoxy)benzamido)-N-(2,3-dichlorobenzyl)piperidine-3-carboxamide hydrochloride [Polar isomer]

Prepared similar to the procedure described in Example 15 but using instead Acid 1 and Amine 2 as a starting material. The title compound was obtained as a thick liquid. ESI-MS: M⁺=665; ^{a}HPLC tᵣₑₜ: 19.56 min.

### Example 36

### (4S)—N-Cyclopropyl-4-(4-(2-(2,6-dichloro-4-methylphenoxy)ethoxy)benzamido)-N-(3-methoxy-2-methylbenzyl)piperidine-3-carboxamide hydrochloride [Non polar isomer]

Prepared similar to the procedure described in Example 14 but using instead Acid 1 and Amine 20 as a starting material. The title compound was obtained as a thick liquid. ESI-MS: M⁺=640; ^{a}HPLC tᵣₑₜ: 18.77 min.

### Example 37

### (4S)—N-Cyclopropyl-4-(4-(2-(2,6-dichloro-4-methylphenoxy)ethoxy)benzamido)-N-(3-methoxy-2-methylbenzyl)piperidine-3-carboxamide hydrochloride [Polar isomer]

Prepared similar to the procedure described in Example 15 but using instead Acid 1 and Amine 20 as a starting material. The title compound was obtained as a thick liquid. ESI-MS: M⁺=640; ^{a}HPLC tᵣₑₜ: 18.38 min.

### Example 38

### (4S)—N-Cyclopropyl-4-(4-(2-(2,6-difluorophenoxy)ethoxy)benzamido)-N-(2,3-dimethylbenzyl)piperidine-3-carboxamide hydrochloride [Polar isomer]

Prepared similar to the procedure described in Example 15 but using instead Acid 3 and Amine 18 as a starting material. The title compound was obtained as a thick liquid. ESI-MS: M⁺=578; ^{a}HPLC tᵣₑₜ: 17.23 min.

### Example 39

### (4S)—N-Cyclopropyl-4-(4-(2-(2,6-difluorophenoxy)ethoxy)benzamido)-N-(2,3-dimethylbenzyl)piperidine-3-carboxamide hydrochloride [Non Polar isomer]

Prepared similar to the procedure described in Example 14 but using instead Acid 3 and Amine 18 as a starting material. The title compound was obtained as a thick liquid. ESI-MS: M⁺=578; ^{d}HPLC tᵣₑₜ: 16.90 min.

### Example 40

### (4S)—N-cyclopropyl-4-(4-(2-(2,6-dichloro-4-methylphenoxy)ethoxy)benzamido)-N-(2,3-dimethylbenzyl)piperidine-3-carboxamide methanesulfonate

(4S)—N-Cyclopropyl-4-(4-(2-(2,6-dichloro-4-methylphenoxy)ethoxy)benzamido)-N-(2,3-dimethylbenzyl)piperidine-3-carboxamide prepared as per reported in Example 26 [1 eq] dissolved in DCM and methane sulfonyl chloride [1.2 eq] was added and mixture was stirred for 1 h. Organic layer was removed in vacuo to afford hygroscopic solid.

### Example 41

### (4S)—N-Cyclopropyl-4-(4-(2-(2,6-dichloro-4-methylphenoxy)ethoxy)benzam ido)-N-(2,3-dimethylbenzyl)piperidine-3-carboxamide 2,2,2-trifluoroacetate

(4S)—N-Cyclopropyl-4-(4-(2-(2,6-dichloro-4-methylphenoxy)ethoxy)benzamido)-N-(2,3-dimethylbenzyl)piperidine-3-carboxamide prepared as per reported in Example 26 [1 eq] dissolved in DCM and trifluoroacetic acid [1.2 eq] was added and mixture was stirred for 1 h. Organic layer was removed in vacuo to afford title compound as white solid m.p.: 198-200° C.

### Example 42

### (4S)—N-Cyclopropyl-4-(4-(2-(2,6-dichloro-4-methylphenoxy)ethoxy)benzamido)-N-(2,3-dimethylbenzyl)piperidine-3-carboxamide bisulfate

(4S)—N-Cyclopropyl-4-(4-(2-(2,6-dichloro-4-methylphenoxy)ethoxy)benzamido)-N-(2,3-dimethylbenzyl)piperidine-3-carboxamide prepared as per reported in Example 26 [1 eq] dissolved in dry acetone and sulfuric acid [0.55 eq] was added and mixture was stirred for 1 h. Organic layer was removed in vacuo to afford title compound as off white solid m.p.: 168-170° C.

### Example 43

### (4S)—N-cyclopropyl-4-(4-(2-(2,6-dichloro-4-methylphenoxy)ethoxy)benzamido)-N-((2,3-dihydrobenzo[b][1,4]dioxin-6-yl)methyl)piperidine-3-carboxamide hydrochloride [Non polar isomer]

Prepared similar to the procedure described in Example 14 but using instead Acid 1 and Amine 12 as a starting material. The title compound was obtained as a semi solid. ESI-MS: M⁺=654; ^{a}HPLC tᵣₑₜ: 16.82 min.

### Example 44

### (4S)—N-cyclopropyl-4-(4-(2-(2,6-dichloro-4-methylphenoxy)ethoxy)benzamido)-N-((2,3-dihydrobenzo[b][1,4]dioxin-6-yl)methyl)piperidine-3-carboxamide hydrochloride [Polar isomer]

Prepared similar to the procedure described in Example 15 but using instead Acid 1 and Amine 12 as a starting material. The title compound was obtained as a semi solid. ESI-MS: =654; ^{a}HPLC tᵣₑₜ: 16.34 min.

### Example 45

### (4S)—N-(benzo[d][1,3]dioxol-4-ylmethyl)-N-cyclopropyl-4-(4-(2-(2,6-dichloro-4-methylphenoxy)ethoxy)benzamido)piperidine-3-carboxamide hydrochloride [Non polar isomer]

Prepared similar to the procedure described in Example 14 but using instead Acid 1 and Amine 16 as a starting material. The title compound was obtained as a semi solid. ESI-MS: M⁺=641; ^{a}HPLC tᵣₑₜ: 17.02 min.

### Example 46

### (4S)—N-(benzo[d][1,3]dioxol-4-ylmethyl)-N-cyclopropyl-4-(4-(2-(2,6-dichloro-4-methylphenoxy)ethoxy)benzamido)piperidine-3-carboxamide hydrochloride [Polar isomer]

Prepared similar to the procedure described in Example 15 but using instead Acid 1 and Amine 16 as a starting material. The title compound was obtained as a semi solid. ESI-MS: M⁺=641; ^{a}HPLC tᵣₑₜ: 16.59 min.

### Example 47

### (4S)—N-cyclopropyl-4-(4-(2-(2,6-dichloro-4-methylphenoxy)ethoxy)benzam ido)-N-((2,3-dihydrobenzo[b][1,4]dioxin-5-yl)methyl)piperidine-3-carboxamide hydrochloride [Non polar isomer]

Prepared similar to the procedure described in Example 14 but using instead Acid 1 and Amine 21 as a starting material. The title compound was obtained as a semi solid. ESI-MS: M⁺=655; ^{a}HPLC tᵣₑₜ: 17.04 min.

### Example 48

### (4S)—N-cyclopropyl-4-(4-(2-(2,6-dichloro-4-methylphenoxy)ethoxy)benzamido)-N-((2,3-dihydrobenzo [b][1,4]dioxin-5-yl)methyl)piperidine-3-carboxamide hydrochloride [Non polar isomer]

Prepared similar to the procedure described in Example 15 but using instead Acid 1 and Amine 21 as a starting material. The title compound was obtained as a semi solid. ESI-MS: M⁺=655; ^{a}HPLC tᵣₑₜ: 16.61 min.

### Example 49

### (4S)—N-cyclopropyl-4-(4-(2-(2,6-dichloro—4-methylphenoxy)ethoxy)benzamido)-N-((1-methoxynaphthalen-2-yl)methyl)piperidine-3-carboxamide hydrochloride [Non polar isomer]

Prepared similar to the procedure described in Example 14 but using instead Acid 1 and Amine 22 as a starting material. The title compound was obtained as a semi solid. ESI-MS: M⁺=676; ^{a}HPLC tᵣₑₜ: 18.07 min.

### Example 50

### (4S)—N-cyclopropyl-4-(4-(2-(2,6-dichloro-4-methylphenoxy)ethoxy)benzam ido)-N-((1-methoxynaphthalen-2-yl)methyl)piperidine-3-carboxamide hydrochloride [Polar isomer]

Prepared similar to the procedure described in Example 15 but using instead Acid 1 and Amine 22 as a starting material. The title compound was obtained as a semi solid. ESI-MS: M⁺=676; ^{a}HPLC tᵣₑₜ: 17.60 min.

### Example 51

### (4S)—N-(2-chlor'o-3-methylbenzyl)-N-cyclopropyl-4-(4-(2-(2,6-dichloro-4-methylphenoxy)ethoxy)benzamido)piperidine-3-carboxamide hydrochloride [Non polar isomer]

Prepared similar to the procedure described in Example 14 but using instead Acid 1 and Amine 17 as a starting material. The title compound was obtained as a semi solid. ESI-MS: M⁺=645; ^{a}HPLC tᵣₑₜ: 18.15 min.

### Example 52

### (4S)—N-(2-chloro-3-methylbenzyl)-N-cyclopropyl-4-(4-(2-(2,6-dichloro-4-methylphenoxy)ethoxy)benzamido)piperidine-3-carboxamide hydrochloride [Polar isomer]

Prepared similar to the procedure described in Example 15 but using instead Acid 1 and Amine 17 as a starting material. The title compound was obtained as a semi solid. ESI-MS: M⁺=645; ^{a}HPLC tᵣₑₜ: 17.69 min.

### Biological Data

In-vitro protocol for the Inhibition of human recombinant renin by the compound of invention
Procedure: The enzymatic in vitro assay was performed in 96 well polypropylene plate (Nunc), using a modified Renin inhibitor screening assay protocol (Cayman, cat no: 10006270). The reaction system comprised assay buffer containing 50 mM Tris-HCL, pH=8.0 & 100 mM sodium chloride, human recombinant rennin (1:20 diluted with fixed activity), synthetic renin substrates (Cayman, cat no: 10006906) (14.4 μM) and different concentrations of renin inhibitors in DMSO in a total reaction system of 100 μl. The entire reaction mixture were incubated at 37° C. for 30 mins and the fluorescence was read at excitation wavelengths of 320-360 nm and emission wavelengths of 490-520 nm. Test compounds efficacy was determined by the percent inhibition of renin activity using Aliskiren (Tekturna) as a reference standard.
   The following table shows the Renin inhibition of selected compounds at 0.1 μM and 0.01 μM concentration.

| Compounds | 0.1 μM | 0.01 μM |
|---|---|---|
| Example 1 | 100 | 88 |
| Example 3 | 43 | * |
| Example 4 | 47 | * |
| Example 5 | 81 | 42 |
| Example 6 | 40 | * |
| Example 8 | 41 | * |
| Example 10 | 44 | * |
| Example 12 | 89 | 43 |
| Example 14 | 66 | 19 |
| Example 15 | 12 | 13 |
| Example 16 | 52 | 11 |
| Example 18 | 56 | 20 |
| Example 20 | 91 | 48 |
| Example 22 | 40 | * |
| Example 24 | 83 | 10 |
| Example 26 | 97 | 67 |
| Example 27 | 38 | * |
| Example 28 | 93 | 60 |
| Example 29 | 65 | 11 |
| Example 30 | 87 | * |
| Example 31 | 91 | 22 |
| Example 33 | 17 | * |
| Example 35 | 95 | 60 |
| Example 37 | 81 | 24 |

* Inactive at tested concentration

Following table represents measured IC₅₀ values of the selected compounds for its inhibition of human recombinant renin.

| Compounds | IC₅₀ nM |
|---|---|
| Example 1 | 16 |
| Example 5 | 39 |
| Example 12 | 64 |
| Example 20 | 13 |
| Example 26 | 21 |
| Example 31 | 37 |
| Example 37 | 17 |

### Anaesthetized Guinea Pig Model

Experimental Procedure: Guinea Pigs were Treated with Furosemide in Drinking Water

(5 mg/kg/day) for four days and intramuscular injection (10 mg/kg) at 18 and 3 hr before experiment. There after they were anaesthetized by using intramuscular injection of xylazine and ketamine (7:70 mg/kg mixture). The left or right jugular vein was exposed and cannulated for intravenous administration of the NCEs. The to left or right carotid artery was exposed and cannulated for recording blood pressure using Biopac system. 0 (vehicle control), 3 and 10 mg of NCEs were administered and change in blood pressure from pretreatment was measured.

Reduction in
M.A.B.P

| Compd. Id. | 3 mg | 10 mg |
|---|---|---|
| Example 1 | 18 | 30 |
| Example 26 | 14 | 28 |
| Example 31 | 8 | 28 |

The pharmaceutical composition is provided by employing conventional techniques. Preferably the composition is in unit dosage form containing an effective amount of the active component, that is, the compounds of formula (1) according to this invention.

The quantity of active component, that is, the compounds of formula (1) according to this invention, in the pharmaceutical composition and unit dosage form thereof may be varied or adjusted widely depending upon the particular application method, the potency of the particular compound and the desired concentration.

We claim:

1. Compound represented by the formula (1), tautomers or pharmaceutically salt thereof 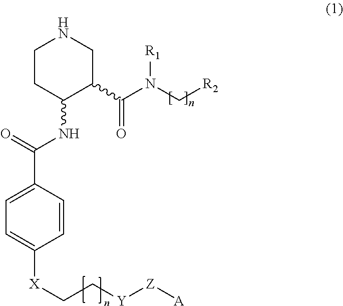
wherein
Z represents either a bond or —CH₂—;
X and Y are each independently selected from the group comprising of —CH₂—, O, and S(O)ₚ; p in each instance when it occurs is independently selected from the integers 0,1,2; n is an integer selected from 0,1,2;
‘A’ is an optionally substituted aryl or a heteroaryl ring wherein the heteroaryl group contains from 1 to 3 heteroatoms selected from O, S, and N; R₁ represents optionally substituted C₁₋C₆ alkyl, or C₃-C₇ cycloalkyl groups; R₂ represents an aryl, or a heteroaryl group, or a hetrocycle group, wherein either of the heteroaryl or heterocycle group contains from 1 to 3 heteroatoms selected from O, S, and N,& wherein each of the said groups can be optionally substituted; R₃ is independently selected from the group comprising of hydrogen, optionally substituted alkyl groups which may be optionally substituted with one, two or three halogen atoms, optionally substituted cycloalkyl, or the groups selected from —C(═O)OR₄ or —C(═O)R₄, wherein R₄ represents optionally substituted groups selected from (C₁-C₄) alkyl, (C₁-C₄)haloalkyl or a cycloalkyl or the group representing R₅NH—C(═O)—(O)—(CH₂)₀₋₄—CH₂₋wherein R₅ is an optionally substituted alkyl or cycloalkyl group;
Alternatively, R₁ and R₂ together with the nitrogen atom attached to R₁ may, together form a saturated, unsaturated or partly saturated single or fused optionally substituted heterocyclic ring which may optionally contain one or more additional hetero atoms selected from nitrogen, oxygen or sulphur or may comprise an —SO— or an —SO₂-group.

2. The compound as claimed in claim 1 wherein the substituent on ‘A’ is selected from OH, CN, halogen, N₃, NO₂, COOH, OCF₂H, CF₃, C₍₁₋₆₎ alkyl, C₍₂₋₆₎ alkenyl, C₍₁₋₆₎ alkoxy, C(O)C₁-C₆ alkyl, S(O)ₚ C₍₁₋₆₎ alkyl or (CH₂)₁₋₂O-alkyl groups.

3. The compound as claimed in claim 1 wherein the substituents on R₂ is selected from the group comprising of alkyl, halogen, alkoxy, —OCF₃, CF₃, hydroxyl-alkyl; alkyl-O—(CH₂)₀₋₄—CH₂—; alkyl-O—(CH₂)₂₋₄—O—; (R₃)₂N—(CH₂)₀₋₄—CH₂-groups.

4. The compound as claimed in claim 1 wherein, when R₁ and R₂ together forms a heterocyclic ring, the heterocyclic group is optionally substituted with groups selected from halogen, hydroxyl, oxide, oxo, cyano, optionally substituted groups selected from haloalkyl, haloalkoxy, C₁-C₈-alkyl, C₁-C₈-alkoxy, C₁-C₈-alkoxy-C₁-C₈-alkyl, C₁-C₈-alkoxy-C₁-C₈-alkoxy, C₁-C₈-alkoxycarbonylamino, C₁-C₈-alkylcarbonylamino, C₁-C₈-alkylamino, N,N-di-C₁-C₈-alkylamino, aryl-C₀-C₄-alkyl, aryloxy-C₀-C₄-alkyl, aryl-C₀-C₄-alkyl-C₁-C₈-alkoxy, aryloxy, C₀-C₄-alkyl-C₁-C₈-alkoxy, heterocyclyl-C₀-C₄-alkyl, heterocyclyloxy-C₀-C₄-alkyl, heterocyclyl-C₀-C₄-alkyl-C₁-C₈-alkoxy or heterocyclyloxy-C₀-C₄-alkyl-C₁-C₈-alkoxy groups.

5. The compound as claimed in claim 1, wherein the Z is bond.

6. The compound as claimed in claim 1, wherein each of X and Y represents oxygen.

7. The compound as claimed in claim 1, wherein the R₁ is selected from hydrogen, alkyl, cycloalkyl groups.

8. The compound as claimed in claim 7 wherein R₁ is selected from cyclopropyl and isopropyl groups.

9. An intermediate as claimed in formula 9 and their isomers 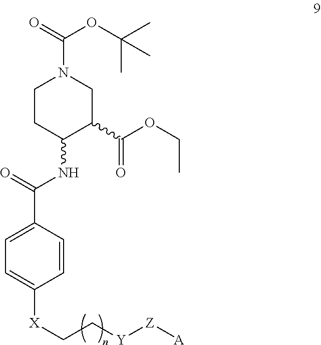
wherein the symbols A, Z, X, n, Y, are as defined in claim 1.

10. An intermediate of formula 4 and their isomers 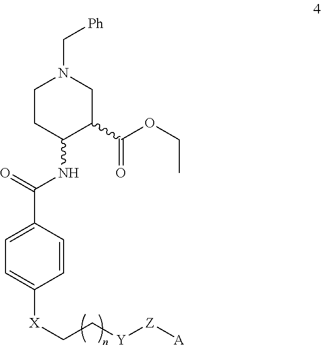
wherein the symbols A, Z, X, n, Y, are as defined in claims 1.

11. The compound as claimed in claim 1 selected from
N-Cyclopropyl-N-(2,3-dichlorobenzyl)-4-(4-(2-(2,6-dichlorophenoxy)ethoxy)benzamido) piperidine-3-carboxamide.
4-(2-(2,6-Dichlorophenoxy)ethoxy)-N-(3-(piperidine-1-carbonyl)piperidin-4-yl)benzamide.
N-Cyclopropyl-4-(4-(2-(2,6-dichlorophenoxy)ethoxy)benzamido)-N-(3-(3-methoxypropoxy)benzyl)piperidine-3-carboxamide.
N-Cyclopropyl-4-(4-(2-(2,6-dichlorophenoxy)ethoxy)benzamido)-N-(3,5-dimethylbenzyl)piperidine-3-carboxamide.
N-Cyclopropyl-4-(4-(2-(2,6-dichloro-4-methylphenoxy)ethoxy)benzamido)-N-(2,4-dichlorobenzyl)piperidine-3-carboxamide.
N-Cyclopropyl-N-(2,4-dichlorobenzyl)-4-(4-(2-(2,6-dichlorophenoxy)ethoxy)benzamido) piperidine-3-carboxamide.
N-Cyclopropyl-4-(4-(2-(2,6-dichlorophenoxy)ethoxy)benzamido)-N-(3,4-dimethoxybenzyl)piperidine-3-carboxamide
N-Cyclopropyl-4-(4-(2-(2,6-dichloro-4-methylphenoxy)ethoxy)benzamido)-N-(3,4-dimethoxybenzyl)piperidine-3-carboxamide
N-Cyclopropyl-4-(4-(2-(2,6-dichloro-4-methylphenoxy)ethoxy)benzamido)-N-(3,4-dimethoxybenzyl)piperidine-3-carboxamide
N-Cyclopropyl-4-(4-(2-(2,6-dichloro-4-methylphenoxy)ethoxy)benzamido)-N-(3,5-dimethylbenzyl)piperidine-3-carboxamide.
N-Cyclopropyl-4-(4-(2-(2,6-dichlorophenoxy)ethoxy)benzamido)-N-(naphthalen-2-ylmethyl)piperidine-3-carboxamide.
N-Cyclopropyl-4-(4-(2-(2,6-dichloro-4-methylphenoxy)ethoxy)benzamido)-N-(naphthalen-2-ylmethyl)piperidine-3-carboxamide.
4-(2-(2,6-Dichloro-4-methylphenoxy)ethoxy)-N-(3-(3-(methoxymethyl)piperidine-1-carbonyl)piperidin-4-yl)benzamide.
(4S)—N-Cyclopropyl-4-(4-(2-(2,6-dichloro-4-methylphenoxy)ethoxy)benzamido)-N-(2,3-dimethoxybenzyl)piperidine-3-carboxamide hydrochloride
(4S)—N-Cyclopropyl-4-(4-(2-(2,6-dichloro-4-methylphenoxy)ethoxy)benzamido)-N-(2,3-dimethoxybenzyl)piperidine-3-carboxamide hydrochloride
(4S)—N-Cyclopropyl-N-(2,3-dimethylbenzyl)-4-(4-(2-((2-methoxybenzyl)oxy)ethoxy)benzamido)piperidine-3-carboxamide
(4S)—N-Cyclopropyl-N-(2,3-dimethylbenzyl)-4-(4-(2-((2-methoxybenzyl)oxy)ethoxy)benzamido)piperidine-3-carboxamide
(4S)—N-Cyclopropyl-4-(4-(2-(2,6-dichloro-4-methylphenoxy)ethoxy)benzamido)-N-(3,4-dimethylbenzyl)piperidine-3-carboxamide hydrochloride
(4S)—N-Cyclopropyl-4-(4-(2-(2,6-dichloro-4-methylphenoxy)ethoxy)benzamido)-N-(2,3-dimethylbenzyl)piperidine-3-carboxamide hydrochloride
(4S)—N-Cyclopropyl-4-(4-(2-(2,6-dichloro-4-methylphenoxy)ethoxy)benzamido)-N-(2,3-dimethylbenzyl)piperidine-3-carboxamide hydrochloride
(4S)—N-Cyclopropyl-4-(4-(2-(2,6-dichloro-4-methylphenoxy)ethoxy)benzamido)-N-(3,4-dimethylbenzyl)piperidine-3-carboxamide hydrochloride
(4R)—N-Cyclopropyl-4-(4-(2-(2,6-dichloro-4-methylphenoxy)ethoxy)benzamido)-N-(3,4-dimethylbenzyl)piperidine-3-carboxamide hydrochloride
to (4R)—N-Cyclopropyl-4-(4-(2-(2,6-dichloro-4-methylphenoxy)ethoxy)benzamido)-N-(3,4-dimethylbenzyl)piperidine-3-carboxamide hydrochloride
(4R)—N-Cyclopropyl-4-(4-(2-(2,6-dichloro-4-methylphenoxy)ethoxy)benzamido)-N-(2,3-dimethylbenzyl)piperidine-3-carboxamide hydrochloride
(4R)—N-Cyclopropyl-4-(4-(2-(2,6-dichloro-4-methylphenoxy)ethoxy)benzamido)-N-(2,3-dimethylbenzyl)piperidine-3-carboxamide hydrochloride
(4S)—N-Cyclopropyl-4-(4-(2-(2,6-dichloro-4-methylphenoxy)ethoxy)benzamido)-N-(2,3-dimethylbenzyl)piperidine-3-carboxamide
(4S)—N-Cyclopropyl-N-(2,3-dimethylbenzyl)-4-(4-(342-methoxybenzypoxy) propoxy)benzamido)piperidine-3-carboxamide hydrochloride
(4S)—N-cyclopropyl-N-(2,3-dimethylbenzyl)-4-(4-(3-((2-methoxybenzyl)oxy)propoxy)benzamido)piperidine-3-carboxamide hydrochloride
(4S)—N-Cyclopropyl-4-(4-(2-(2,6-dichloro-4-methylphenoxy)ethoxy)benzamido)-N-(2-fluorobenzyl)piperidine-3-carboxamide hydrochloride.
(4S)—N-(2-Chlorobenzyl)-N-cyclopropyl-4-(4-(2-(2,6-dichloro-4-methylphenoxy)ethoxy)benzamido)piperidine-3-carboxamide hydrochloride
(4S)—N-Cyclopropyl-4-(4-(2-(2,6-dichloro-4-methylphenoxy)ethoxy)benzamido)-N-(2-methoxybenzyl)piperidine-3-carboxamide hydrochloride
(4S)—N-Cyclopropyl-4-(4-(2-(2,6-dichloro-4-methylphenoxy)ethoxy)benzamido)-N-(2-methoxybenzyl)piperidine-3-carboxamide hydrochloride
(4S)—N-Cyclopropyl-N-(2-methoxybenzyl)-4-(4-(3-((2-methoxybenzyl)oxy)propoxy)benzamido)piperidine-3-carboxamide hydrochloride
(4S)—N-Cyclopropyl-N-(2-methoxybenzyl)-4-(4-(3-((2-methoxybenzyl)oxy)propoxy)benzamido)piperidine-3-carboxamide hydrochloride
(4S)—N-Cyclopropyl-4-(4-(2-(2,6-dichloro-4-methylphenoxy)ethoxy)benzamido)-N-(2,3-dichlorobenzyl)piperidine-3-carboxamide hydrochloride
(4S)—N-Cyclopropyl-4-(4-(2-(2,6-dichloro-4-methylphenoxy)ethoxy)benzamido)-N-(3-methoxy-2-methylbenzyl)piperidine-3-carboxamide hydrochloride
(4S)—N-cyclopropyl-4-(4-(2-(2,6-dichloro-4-methylphenoxy)ethoxy)benzamido)-N-(3-methoxy-2-methylbenzyl)piperidine-3-carboxamide hydrochloride
(4S)N-Cyclopropyl-4-(4-(2-(2,6-difluorophenoxy)ethoxy)benzamido)-N-(2,3-dimethylbenzyl)piperidine-3-carboxamide hydrochloride
(4S)—N-Cyclopropyl-4-(4-(2-(2,6-difluorophenoxy)ethoxy)benzamido)-N-(2,3-dimethylbenzyl)piperidine-3-carboxamide hydrochloride
(4S)—N-Cyclopropyl-4-(4-(2-(2,6-dichloro-4-methylphenoxy)ethoxy)benzamido)-N-(2,3-dimethylbenzyl)piperidine-3-carboxamide methanesulfonate.
(4S)—N-Cyclopropyl-4-(4-(2-(2,6-dichloro-4-methylphenoxy)ethoxy)benzamido)-N-(2,3-dimethylbenzyl)piperidine-3-carboxamide 2,2,2-trifluoroacetate.
(4S)—N-Cyclopropyl-4-(4-(2-(2,6-dichloro-4-methylphenoxy)ethoxy)benzamido)-N-(2,3-dimethylbenzyl)piperidine-3-carboxamide bisulfate.
(4S)—N-cyclopropyl-4-(4-(2-(2,6-dichloro-4-methylphenoxy)ethoxy)benzamido)-N-((2,3-dihydrobenzo[b][1,4]dioxin-6-yl)methyl)piperidine-3-carboxamide hydrochloride
(4S)—N-cyclopropyl-4-(4-(2-(2,6-dichloro-4-methylphenoxy)ethoxy)benzamido)-N-((2,3-dihydrobenzo[b][1,4]dioxin-6-yl)methyl)piperidine-3-carboxamide hydrochloride
(4S)—N-(benzo[d][1,3]dioxol-4-ylmethyl)-N-cyclopropyl-4-(4-(2-(2,6-dichloro-4-methylphenoxy)ethoxy)benzamido)piperidine-3-carboxamide hydrochloride
(4S)—N-(benzo[d][1,3]dioxol-4-ylmethyl)-N-cyclopropyl-4-(4-(2-(2,6-dichloro-4-methylphenoxy)ethoxy)benzamido)piperidine-3-carboxamide hydrochloride
(4S)—N-cyclopropyl-4-(4-(2-(2,6-dichloro-4-methylphenoxy)ethoxy)benzamido)-N-((2,3-dihydrobenzo[b][1,4]dioxin-5-yl)methyl)piperidine-3-carboxamide hydrochloride.
(4S)—N-cyclopropyl-4-(4-(2-(2,6-dichloro-4-methylphenoxy)ethoxy)benzamido)-N-((2,3-dihydrobenzo[b][1,4]dioxin-5-yl)methyl)piperidine-3-carboxamide hydrochloride
(4S)—N-cyclopropyl-4-(4-(2-(2,6-dichloro-4-methylphenoxy)ethoxy)benzamido)-N-((1-methoxynaphthalen-2-yl)methyl)piperidine-3-carboxamide hydrochloride.
(4S)—N-cyclopropyl-4-(4-(2-(2,6-dichloro-4-methylphenoxy)ethoxy)benzamido)-N-((1-methoxynaphthalen-2-yl)methyl)piperidine-3-carboxamide hydrochloride.
(4S)—N-(2-chloro-3-methylbenzyl)-N-cyclopropyl-4-(4-(2-(2,6-dichloro-4-methylphenoxy)ethoxy)benzamido)piperidine-3-carboxamide hydrochloride.
(4S)—N-(2-chloro-3-methylbenzyl)-N-cyclopropyl-4-(4-(2-(2,6-dichloro-4-methylphenoxy)ethoxy)benzamido)piperidine-3-carboxamide hydrochloride

12. A pharmaceutical composition comprising a therapeutically effective amount of a compound of formula 1 as claimed in any of the preceding claims and a pharmaceutically acceptable carrier diluent or excipients.

13. A pharmaceutical composition according to claim 12 used for the treatment of diseases wherein the renin enzyme has a patho physiological function.

14. Use of a compound of claim 1, or a composition according to claim 12, for the manufacture of a medicament for the treatment or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system.
